(19) United States
(12) Patent Application Publication
(10) Pub. No.: US 2026/0057520 A1
(43) Pub. Date: Feb. 26, 2026

(54) METHODS AND SYSTEMS FOR EVALUATING TUMOR HETEROGENEITY USING HISTOPATHOLOGY IMAGING

(71) Applicant: Foundation Medicine, Inc., Boston, MA (US)

(72) Inventors: James PAO, Boston, MA (US); Karthikeyan MURUGESAN, Boston, MA (US)

(73) Assignee: Foundation Medicine, Inc., Boston, MA (US)

(21) Appl. No.: 19/375,673

(22) Filed: Oct. 31, 2025

Related U.S. Application Data
(63) Continuation of application No. PCT/US2024/027188, filed on May 1, 2024.
(60) Provisional application No. 63/463,464, filed on May 2, 2023.

(51) Int. Cl. G06T 7/00; G06T 7/11; G06V 10/25; G06V 10/70; G16H 50/20

(52) U.S. Cl. CPC G06T 7/0014; G06T 7/11; G16H 50/20; G06T 2207/20081; G06T 2207/20084; G06T 2207/30096; G06V 10/25; G06V 10/70; G06V 2201/03

(57) ABSTRACT

Methods for determining tumor heterogeneity are described. The methods may comprise, for example, obtaining a medical image associated with the sample from a subject; identifying a plurality of patches from the medical image; identifying a plurality of patch groups, wherein each patch group comprises one or more patches of the plurality of patches and corresponds to a region of interest in the medical image; inputting each patch group into a trained machine learning model to generate a plurality of genomic alteration predictions corresponding to the plurality of patch groups, wherein the plurality of genomic alteration predictions is related to the presence of one or more genomic alterations in each of the input patch groups; and generating the phenotypic tumor heterogeneity score by comparing the plurality of genomic alteration predictions.

### CROSS-REFERENCE TO RELATED APPLICATION

This application is a continuation application of International Application No. PCT/US2024/027188, filed internationally on May 1, 2024, which claims the priority benefit of U.S. Provisional Patent Application Ser. No. 63/463,464, filed May 2, 2023, the disclosures of which are herein incorporated by reference in their entirety.

### FIELD OF THE INVENTION

The present disclosure relates generally to methods and systems for analyzing genomic histopathology image data, and more specifically to methods and systems for evaluating tumor heterogeneity using histopathology image data.

### BACKGROUND

Cancer biology may follow a Darwinian evolutionary paradigm, where a cancer may originate with truncal alterations but then have subclones arise that compete not only against the healthy tissue but also against each other for survival and proliferation. A driver alteration may first occur to allow for the initiation and proliferation of the cancer. As the cancer progresses, additional mutations may occur in individual cancer cells and generate subclones within the tumor that can possess differing molecular profiles as well as differing profiles of progression and therapeutic response. The selective advantage of certain subclones within a cancer may help explain the usually finite duration of response for various treatments, and the inevitable recurrence of certain diseases. Sub-clones that are highly susceptible to treatments may be quickly eliminated, thus readjusting the equilibrium of the tumor microenvironment and allowing for treatment-resistance subclones to gain a competitive advantage and proliferate in the absence or reduction of competing subclones.

Accordingly, tumors may be constituted of geographically separate regions in which the molecular profiles may differ, whether across multiple driver alterations (e.g. in the case of colliding primaries) or across secondary co-occurring alterations. Although the single oncogene-driven model of cancer alone has been effective in establishing clinical guidelines, there is increasing evidence that certain alterations that co-occur with oncogenic drivers are highly influential in shaping the tumor histology and microenvironment, which may have direct consequence on treatment efficacies. Within lung adenocarcinoma (LUAD) cancers, KRAS-driven tumors are often highly varied in histology as well as immunohistochemistry results; for example, when lacking NKX2-1, KRAS tumors present in the mucinous histology but present as solid growth patterns when NKX2-1 is expressed. Another oncogene, EGFR, is a truncal alteration that may also have other co-occurring clonal alterations that may affect the disease course. Additionally, EGFR mutated LUAD cancers are co-mutated with genes such as CTNNB1 and PIK3CA, both of which are commonly encountered EGFR co-mutations in late stage LUADs. In such cases, the co-mutations to the EGFR oncogene driver could be sub-clonal and vary across different geographical regions of the tumor. If so, and if the co-mutations influence changes in the standard EGFR-mutated histology or tumor microenvironment, then there may be visual distinction between regions of a given tissue specimen.

Thus, it is desirable to develop techniques for evaluating tumor heterogeneity using histopathology images, which may, for example, have significant clinical implications and give insight into the duration and effectiveness of therapeutic responses.

### BRIEF SUMMARY OF THE INVENTION

Methods and systems for evaluating tumor heterogeneity using histopathology images are described. Some embodiments of the present disclosure can determine a phenotypic/morphological tumor heterogeneity score associated a tumor sample by comparing the mutation status of one or more genomic alterations across multiple regions of interest in a histopathology image of the tumor sample. An exemplary system can obtain an image (e.g., a medical image) associated with a sample from an individual. The system can identify a plurality of patches from the medical image and then identify a plurality of patch groups, each patch group comprising one or more patches of the plurality of patches and corresponds to a region of interest in the medical image. The system can input each patch group into a trained machine learning model to generate a plurality of genomic alteration predictions corresponding to the plurality of patch groups. The plurality of genomic alteration predictions includes the presence of one or more genomic alterations in each of the input patch groups. The system can then generate a phenotypic tumor heterogeneity score by comparing the plurality of genomic alteration predictions. In some embodiments, the model is configured to predict the mutation status of a single alteration (e.g., EGFR only), and the system can then generate an alteration-specific clonality estimate and a subsequent heterogeneity score. In some embodiments, the model is configured to predict the mutational statuses for a multitude of genes rather than a single one.

Some embodiments of the present disclosure can determine a phenotypic tumor heterogeneity score associated a tumor sample by comparing a tumor microenvironment profile across multiple regions of interest in a histopathology image of the tumor sample. An exemplary system can obtain a medical image associated with the sample from the individual and input the medical image into a trained machine learning model to identify a plurality of phenotypic features in the medical image. The system can identify a plurality of patches from the medical image and then identify a plurality of patch groups. Each patch group comprises one or more patches of the plurality of patches and corresponds to a region of interest in the medical image. The system can obtain, based on the plurality of phenotypic features, a plurality of microenvironment profiles corresponding to the plurality of patch groups and generate a phenotypic tumor heterogeneity score by comparing the plurality of microenvironment profiles.

Some embodiments of the present disclosure can determine a phenotypic tumor heterogeneity score associated a tumor sample by comparing numerical representations (e.g., embeddings) corresponding to multiple regions of interest in a histopathology image of the tumor sample. An exemplary system can obtain an image (e.g., a medical image) associated with a sample from an individual. The system can identify a plurality of patches from the medical image and then identify a plurality of patch groups, each patch group comprising one or more patches of the plurality of patches and corresponds to a region of interest in the medical image. The system can input each patch group into a trained machine learning model to generate a plurality of embeddings corresponding to the plurality of patch groups and generate a phenotypic tumor heterogeneity score by comparing the plurality of embeddings.

In any of the embodiments above, the phenotypic tumor heterogeneity score can be combined with a genomic tumor heterogeneity score to generate a composite tumor heterogeneity score. In some embodiments, the composite score can provide a view on tumor heterogeneity that uses both bulk sequencing but also captures orthogonal information from imaging that may provide spatial insight in a way that may be difficult with bulk sequencing derived comprehensive genomic profiling (CGP). In some embodiments, the composite score can be optimized and tuned for effectiveness at stratifying patient populations on outcomes. For example, the system can identify one or more threshold composite scores for stratifying patients based on the outcome data (e.g., response to one or more treatments, survival, relapse, etc.).

The genomic tumor heterogeneity score may be obtained from sequencing data associated with the same sample depicted in the image (e.g., whole slide image), a different sample of the subject, and/or a sample from a comparable subject. In some embodiments, the genomic tumor heterogeneity score comprises a bulk sequencing genomics-based tumor heterogeneity score that uses a cancer cell fraction (CCF) estimation. CCF may be defined as the fraction of tumor cells from the sequenced specimen carrying a set of short variants. In some embodiments, the CCF of every short variant (SV) detected in the specimen is estimated. The estimated CCFs can be used to generate the genomic heterogeneity score, which may be calculated as the ratio of the median CCF of all short variants to the quartile coefficient of dispersion (QCD) of all short variants. In some embodiments, the genomic tumor heterogeneity score is calculated using data derived from a tissue specimen's single region bulk tumor targeted exome assay. Additional details of exemplary genomic tumor heterogeneity score can be found in, for example, PCT Application No. PCT/US2023/064598, titled “CHARACTERIZATION OF TUMOR HETEROGENEITY AS A PROGNOSTIC BIOMARKER,” the content of which is incorporated herein by reference.

Measuring the heterogeneity of tumors based on a single, pre-treatment tissue biopsy according to some embodiments in the present disclosure may give insight into the duration of therapeutic response based on Darwinian evolution principles of tumors under a given selection pressure. For example, tumors with little genomic diversity and from an evolutionary standpoint having lesser overall fitness may require more time to accumulate the necessary mutations required to achieve resistance. In contrast, tumors with higher genomic diversity may require less time to accumulate and select for a resistance mutation and therefore associate with shorter response durations. In this way, measuring the baseline tumor's heterogeneity may serve to predict the duration of response to a particular therapy.

Accordingly, understanding tumor heterogeneity using embodiments of the present disclosure can allow development of prognostic biomarkers to provide insight to duration of therapeutic response (e.g., at tissue baseline) and development of combinatorial treatment options, as well as providing opportunity to recommend longitudinal monitoring based clinical interventions. For example, NSCLC patients who progress on Osimertinib (Third generation TKI) due to acquired resistance from a MET amplification can be treated with a combination of Osimertinib and MET inhibitor. As another example, patients with a TP53 mutation may be treated with Osimertinib plus Carboplatin/Etoposide; patients with RAS/RAF/MAPK alterations may be treated with Osimertinib plus a MEK inhibitor; and patients EGFR C797 alterations may be treated with Osimertinib plus a fourth-generation EGFR TKI. Predicting the duration of therapeutic benefit at baseline can have significant clinical implications for combinatorial treatment strategies, next-line therapy decisions, and tailoring novel monitoring capabilities based on likelihood of relapse.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

An exemplary method for determining a composite tumor heterogeneity score in a sample from a subject comprises: providing a plurality of nucleic acid molecules obtained from the sample from the subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, at one or more processors, sequence read data for the plurality of sequence reads; generating, using the one or more processors, a genomic tumor heterogeneity score based on the sequence read data; obtaining a medical image associated with the sample; identifying a plurality of patches from the medical image; identifying a plurality of patch groups, wherein each patch group comprises one or more patches of the plurality of patches and corresponds to a region of interest in the medical image; inputting each patch group into a trained machine learning model to generate a plurality of genomic alteration predictions corresponding to the plurality of patch groups, wherein the plurality of genomic alteration predictions is related to the presence of one or more genomic alterations in each of the input patch groups; generating a phenotypic tumor heterogeneity score by comparing the plurality of genomic alteration predictions; and calculating the composite tumor heterogeneity score based on the phenotypic tumor heterogeneity score and the genomic tumor heterogeneity score.

In some embodiments, the medical image comprises a histopathology image.

In some embodiments, the composite tumor heterogeneity score is a weighted average or a binarization of the phenotypic tumor heterogeneity score and the genomic tumor heterogeneity score.

In some embodiments, the method further comprises: identifying a combination of treatments for the subject based on the composite tumor heterogeneity score.

In some embodiments, the method further comprises: determining a monitoring plan for the subject based on the phenotypic tumor heterogeneity score.

In some embodiments, the method further comprises: if the composite tumor heterogeneity score is less than or equal to a threshold tumor heterogeneity score, predicting the subject to have a longer duration of therapeutic response to an anti-cancer therapy; and if the composite tumor heterogeneity score is more than the threshold tumor heterogeneity score, predicting the subject to have a shorter duration of therapeutic response to the anti-cancer therapy.

In some embodiments, the method further comprises: if the composite tumor heterogeneity score is less than or equal to a threshold tumor heterogeneity score, predicting the subject to have a higher survival rate if treated with an anti-cancer therapy; and if the composite tumor heterogeneity score is more than the threshold tumor heterogeneity score, predicting the subject to have a lower survival rate if treated with the anti-cancer therapy.

In some embodiments, the subject is suspected of having or is determined to have cancer.

In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some embodiments, the method further comprises treating the subject with an anti-cancer therapy.

In some embodiments, the anti-cancer therapy comprises a targeted anti-cancer therapy.

In some embodiments, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some embodiments, the method further comprises obtaining the sample from the subject.

In some embodiments, the sample comprises a tissue biopsy sample, a liquid biopsy sample, or a normal control.

In some embodiments, the sample is a liquid biopsy sample and comprises blood, plasma, cerebrospinal fluid, sputum, stool, urine, or saliva.

In some embodiments, the sample is a liquid biopsy sample and comprises circulating tumor cells (CTCs).

In some embodiments, the sample is a liquid biopsy sample and comprises cell-free DNA (cfDNA), circulating tumor DNA (ctDNA), or any combination thereof.

In some embodiments, the plurality of nucleic acid molecules comprises a mixture of tumor nucleic acid molecules and non-tumor nucleic acid molecules.

In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample.

In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In some embodiments, the one or more adapters comprise amplification primers, flow cell adaptor sequences, substrate adapter sequences, or sample index sequences.

In some embodiments, the captured nucleic acid molecules are captured from the amplified nucleic acid molecules by hybridization to one or more bait molecules.

In some embodiments, the one or more bait molecules comprise one or more nucleic acid molecules, each comprising a region that is complementary to a region of a captured nucleic acid molecule.

In some embodiments, amplifying nucleic acid molecules comprises performing a polymerase chain reaction (PCR) amplification technique, a non-PCR amplification technique, or an isothermal amplification technique.

In some embodiments, the sequencing comprises use of a massively parallel sequencing (MPS) technique, whole genome sequencing (WGS), whole exome sequencing, targeted sequencing, direct sequencing, or Sanger sequencing technique.

In some embodiments, the sequencing comprises massively parallel sequencing, and the massively parallel sequencing technique comprises next generation sequencing (NGS).

In some embodiments, the sequencer comprises a next generation sequencer.

In some embodiments, one or more of the plurality of sequencing reads overlap one or more gene loci within one or more subgenomic intervals in the sample.

In some embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In some embodiments, the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLDI, POLE, PPARG, PPP2RIA, PPP2R2A, PRDM1, PRKAR1A, PRKC1, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

In some embodiments, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

In some embodiments, the method further comprises generating, by the one or more processors, a report indicating the presence or absence of the genomic alteration.

In some embodiments, the method further comprises transmitting the report to a healthcare provider.

In some embodiments, the report is transmitted via a computer network or a peer-to-peer connection.

An exemplary method for determining a phenotypic tumor heterogeneity score in a sample from a subject comprises: obtaining a medical image associated with the sample from the subject; identifying a plurality of patches from the medical image; identifying a plurality of patch groups, wherein each patch group comprises one or more patches of the plurality of patches and corresponds to a region of interest in the medical image; inputting each patch group into a trained machine learning model to generate a plurality of genomic alteration predictions corresponding to the plurality of patch groups, wherein the plurality of genomic alteration predictions is related to the presence of one or more genomic alterations in each of the input patch groups; and generating the phenotypic tumor heterogeneity score by comparing the plurality of genomic alteration predictions.

In some embodiments, the medical image comprises a histopathology image.

In some embodiments, the method further comprises obtaining a genomic tumor heterogeneity score based on sequence read data associated with the sample; and calculating a composite tumor heterogeneity score based on the phenotypic tumor heterogeneity score and the genomic tumor heterogeneity score.

In some embodiments, the composite tumor heterogeneity score is a weighted average or a binarization of the phenotypic tumor heterogeneity score and the genomic tumor heterogeneity score.

In some embodiments, the method further comprises: identifying a combination of treatments for the subject based on the composite tumor heterogeneity score.

In some embodiments, the method further comprises: determining a monitoring plan for the subject based on the phenotypic tumor heterogeneity score before sequencing the sample from the subject.

In some embodiments, the method further comprises: if the composite tumor heterogeneity score is less than or equal to a threshold tumor heterogeneity score, predicting the subject to have a longer duration of therapeutic response to an anti-cancer therapy; and if the composite tumor heterogeneity score is more than the threshold tumor heterogeneity score, predicting the subject to have a shorter duration of therapeutic response to the anti-cancer therapy.

In some embodiments, the method further comprises: if the composite tumor heterogeneity score is less than or equal to the threshold tumor heterogeneity score, predicting the subject to have a higher survival rate if treated with an anti-cancer therapy; and if the composite tumor heterogeneity score is more than the threshold tumor heterogeneity score, predicting the subject to have a lower survival rate if treated with the anti-cancer therapy.

In some embodiments, the method further comprises: identifying a plurality of regions of interest in the medical image.

In some embodiments, identifying the plurality of regions of interest comprises: inputting the medical image into a trained segmentation machine learning model to identify a tumor tissue depicted in the medical image.

In some embodiments, the plurality of regions of interest corresponds to different visually distinct portions of the tumor tissue in the medical image.

In some embodiments, the plurality of regions of interest corresponds to different local neighborhoods of the tumor tissue in the medical image.

In some embodiments, the trained machine learning model is configured to receive a given input patch group and output a single prediction of the presence of a single genomic alteration in the given input patch group.

In some embodiments, the single genomic alteration is: an epidermal growth factor receptor (EGFR) driver mutation. a BRAF driver mutation, a MET driver mutation, an ERBB2 (HER2) driver mutation, a PIK3CA driver mutation, a KRAS driver mutation, an NRAS driver mutation, a BRCA1 driver mutation, a BRCA2 driver mutation, an ATM driver mutation, an FGFR2 driver mutation, a driver mutation in a homologous recombination repair (HRR) gene, an NTRK1 driver mutation, an NTRK2 driver mutation, an NTRK3 driver mutation, an RET driver mutation, an ALK mutation, or a ROS1 mutation.

In some embodiments, generating the phenotypic tumor heterogeneity score comprises: assigning the phenotypic tumor heterogeneity score to a first value indicative of lower tumor heterogeneity if the plurality of genomic alteration predictions all indicates the presence of the single genomic alteration; and assigning the phenotypic tumor heterogeneity score to a second value indicative of higher tumor heterogeneity if some of the plurality of genomic alteration predictions indicate the presence of the single genomic alteration and some of the plurality of genomic alteration predictions do not indicate the presence of the single genomic alteration.

In some embodiments, generating the phenotypic tumor heterogeneity score comprises: calculating a variance value based on the plurality of genomic alteration predictions; assigning the phenotypic tumor heterogeneity score to a first value indicative of lower tumor heterogeneity if the variance value is lower than or equal to a predefined threshold; and assigning the phenotypic tumor heterogeneity score to a second value indicative of higher tumor heterogeneity if the variance value is higher than a predefined threshold.

In some embodiments, the trained machine learning model is configured to receive a given input patch group and output a plurality of predictions of the presence of a plurality of genomic alterations in the given patch group.

In some embodiments, the plurality of genomic alterations comprises: an epidermal growth factor receptor (EGFR) driver mutation. A BRAF driver mutation, a MET driver mutation, an ERBB2 (HER2) driver mutation, a PIK3CA driver mutation, a KRAS driver mutation, an NRAS driver mutation, a BRCA1 driver mutation, a BRCA2 driver mutation, an ATM driver mutation, an FGFR2 driver mutation, a driver mutation in a homologous recombination repair (HRR) gene, an NTRK1 driver mutation, an NTRK2 driver mutation, an NTRK3 driver mutation, an RET driver mutation, an ALK mutation, a ROS1 mutation, or any combination thereof.

In some embodiments, the sum of the plurality of predictions is configured to be 1.

In some embodiments, the plurality of predictions is independent of each other.

In some embodiments, generating the phenotypic tumor heterogeneity score comprises: generating a plurality of phenotypic tumor heterogeneity sub-scores corresponding to the plurality of genomic alterations, and calculating the phenotypic tumor heterogeneity score based on the plurality of phenotypic tumor heterogeneity sub-scores.

In some embodiments, the trained machine learning model comprises: a convolutional neural network model, a vision transformer model, or any combination thereof.

In some embodiments, the convolutional neural network model comprises an attention mechanism.

An exemplary method for determining a phenotypic tumor heterogeneity score in a sample from a subject comprises: obtaining a medical image associated with the sample from the subject; inputting the medical image into a trained machine learning model to identify a plurality of phenotypic features in the medical image; identifying a plurality of patches from the medical image; identifying a plurality of patch groups, wherein each patch group comprises one or more patches of the plurality of patches and corresponds to a region of interest in the medical image; obtaining, based on the plurality of phenotypic features, a plurality of microenvironment profiles corresponding to the plurality of patch groups; and generating the phenotypic tumor heterogeneity score by comparing the plurality of microenvironment profiles.

In some embodiments, the medical image comprises a histopathology image.

In some embodiments, the method further comprises: obtaining a genomic tumor heterogeneity score based on sequence read data associated with the sample from the subject; and calculating a composite tumor heterogeneity score based on the phenotypic tumor heterogeneity score and the genomic tumor heterogeneity score.

In some embodiments, the composite tumor heterogeneity score is a weighted average or a binarization of the phenotypic tumor heterogeneity score and the genomic tumor heterogeneity score.

In some embodiments, the method further comprises: identifying a combination of treatments for the subject based on the composite tumor heterogeneity score.

In some embodiments, the method further comprises: determining a monitoring plan for the subject based on the phenotypic tumor heterogeneity score before sequencing the sample from the subject.

In some embodiments, the method further comprises: if the composite tumor heterogeneity score is less than or equal to a threshold tumor heterogeneity score, predicting the subject to have a longer duration of therapeutic response to an anti-cancer therapy; and if the composite tumor heterogeneity score is more than the threshold tumor heterogeneity score, predicting the subject to have a shorter duration of therapeutic response to the anti-cancer therapy.

In some embodiments, the method further comprises: if the composite tumor heterogeneity score is less than or equal to the threshold tumor heterogeneity score, predicting the subject to have a higher survival rate if treated with an anti-cancer therapy; and if the composite tumor heterogeneity score is more than the threshold tumor heterogeneity score, predicting the subject to have a lower survival rate if treated with the anti-cancer therapy.

In some embodiments, the method further comprises: identifying a plurality of regions of interest in the medical image.

In some embodiments, identifying the plurality of regions of interest comprises: inputting the medical image into a trained segmentation machine learning model to identify a tumor sample depicted in the medical image.

In some embodiments, the plurality of regions of interest corresponds to different visually distinct portions of the tumor sample in the medical image.

In some embodiments, the plurality of regions of interest corresponds to different local neighborhoods of the tumor sample in the medical image.

In some embodiments, the plurality of phenotypic features comprises: polarity, concavity, texture, shape, and/or size of one or more cells in the sample.

In some embodiments, each microenvironment profile of the plurality of microenvironment profiles comprises: density and/or infiltration of one or more immune cells, a tumor histology type, a tumor cell type, stroma, a normal cell type, necrosis, an immune cell type, a group-to-group ratio, or any combination thereof.

In some embodiments, the trained machine learning model comprises: a segmentation model, a classification model, an object detection model, or any combination thereof.

In some embodiments, generating the phenotypic tumor heterogeneity score comprises: calculating a variance value based on the plurality of microenvironment profiles; assigning the phenotypic tumor heterogeneity score to a first value indicative of lower tumor heterogeneity if the variance value is lower than or equal to a predefined threshold; and assigning the phenotypic tumor heterogeneity score to a second value indicative of higher tumor heterogeneity if the variance value is higher than a predefined threshold.

An exemplary method of determining a phenotypic tumor heterogeneity score in a sample from a subject comprises: obtaining a medical image associated with the sample from the subject; identifying a plurality of patches from the medical image; identifying a plurality of patch groups, wherein each patch group comprises one or more patches of the plurality of patches and corresponds to a region of interest in the medical image; inputting each patch group into a trained machine learning model to generate a plurality of embeddings corresponding to the plurality of patch groups; and generating the phenotypic tumor heterogeneity score by comparing the plurality of embeddings.

In some embodiments, the medical image comprises a histopathology image.

In some embodiments, the method further comprises: obtaining a genomic tumor heterogeneity score based on sequence read data associated with the sample from the subject; and calculating a composite tumor heterogeneity score based on the phenotypic tumor heterogeneity score and the genomic tumor heterogeneity score.

In some embodiments, the composite tumor heterogeneity score is a weighted average or a binarization of the phenotypic tumor heterogeneity score and the genomic tumor heterogeneity score.

In some embodiments, the method further comprises: identifying a combination of treatments for the subject based on the composite tumor heterogeneity score.

In some embodiments, the method further comprises: determining a monitoring plan for the subject based on the phenotypic tumor heterogeneity score before sequencing the sample from the subject.

In some embodiments, the method further comprises: if the composite tumor heterogeneity score is less than or equal to a threshold tumor heterogeneity score, predicting the subject to have a longer duration of therapeutic response to an anti-cancer therapy; and if the composite tumor heterogeneity score is more than the threshold tumor heterogeneity score, predicting the subject to have a shorter duration of therapeutic response to the anti-cancer therapy.

In some embodiments, the method further comprises: if the composite tumor heterogeneity score is less than or equal to the threshold tumor heterogeneity score, predicting the subject to have a higher survival rate if treated with an anti-cancer therapy; and if the composite tumor heterogeneity score is more than the threshold tumor heterogeneity score, predicting the subject to have a lower survival rate if treated with the anti-cancer therapy.

In some embodiments, the method further comprises: identifying a plurality of regions of interest in the medical image.

In some embodiments, identifying the plurality of regions of interest comprises: inputting the medical image into a trained segmentation machine learning model to identify a tumor sample depicted in the medical image.

In some embodiments, the plurality of regions of interest corresponds to different visually distinct portions of the tumor sample in the medical image.

In some embodiments, the plurality of regions of interest corresponds to different local neighborhoods of the tumor sample in the medical image.

In some embodiments, generating the phenotypic tumor heterogeneity score comprises: calculating a plurality of Euclidean distances, each Euclidean distance representing a difference between two embeddings of the plurality of embeddings.

In some embodiments, generating the phenotypic tumor heterogeneity score comprises: calculating a variance value based on the plurality of Euclidean distances.

In some embodiments, generating the phenotypic tumor heterogeneity score comprises: selecting one or more feature values from each embedding of the plurality of embeddings for comparison.

In some embodiments, the trained machine learning model comprises an unsupervised model.

In some embodiments, the trained machine learning model comprises an autoencoder, a computer vision model, or any combination thereof.

An exemplary method for diagnosing a disease comprises: diagnosing that a subject has the disease based on a determination of a phenotypic tumor heterogeneity score or a composite tumor heterogeneity score for a sample from the subject, wherein the phenotypic tumor heterogeneity score or the composite tumor heterogeneity score is determined according to the method of any one of the techniques described herein.

An exemplary method of selecting an anti-cancer therapy comprises: responsive to determining a phenotypic tumor heterogeneity score or a composite tumor heterogeneity score for a sample from a subject, selecting an anti-cancer therapy for the subject, wherein the phenotypic tumor heterogeneity score or the composite tumor heterogeneity score is determined according to the method of any one of the techniques described herein.

An exemplary method of treating a cancer in a subject comprises: responsive to determining a phenotypic tumor heterogeneity score or a composite tumor heterogeneity score for a sample from the subject, administering an effective amount of an anti-cancer therapy to the subject, wherein the phenotypic tumor heterogeneity score or the composite tumor heterogeneity score is determined according to the method of any one of the techniques described herein.

An exemplary method for monitoring cancer progression or recurrence in a subject comprises: determining a first phenotypic tumor heterogeneity score or a first composite tumor heterogeneity score in a first sample obtained from the subject at a first time point according to the method of any one of the techniques described herein; determining a second phenotypic tumor heterogeneity score or a second composite tumor heterogeneity score in a second sample obtained from the subject at a second time point; and comparing the first phenotypic tumor heterogeneity score or the first composite tumor heterogeneity score to the second phenotypic tumor heterogeneity score or the second composite tumor heterogeneity score, thereby monitoring the cancer progression or recurrence.

In some embodiments, the second phenotypic tumor heterogeneity score or the second composite tumor heterogeneity score for the second sample is determined according to the method of any one of the techniques described herein.

In some embodiments, the method further comprises selecting an anti-cancer therapy for the subject in response to the cancer progression.

In some embodiments, the method further comprises administering an anti-cancer therapy to the subject in response to the cancer progression.

In some embodiments, the method further comprises adjusting an anti-cancer therapy for the subject in response to the cancer progression.

In some embodiments, the method further comprises adjusting a dosage of the anti-cancer therapy or selecting a different anti-cancer therapy in response to the cancer progression.

In some embodiments, the method further comprises administering the adjusted anti-cancer therapy to the subject.

In some embodiments, the first time point is before the subject has been administered an anti-cancer therapy, and wherein the second time point is after the subject has been administered the anti-cancer therapy.

In some embodiments, the subject has a cancer, is at risk of having a cancer, is being routine tested for cancer, or is suspected of having a cancer.

In some embodiments, the cancer is a solid tumor.

In some embodiments, the cancer is a hematological cancer.

In some embodiments, the anti-cancer therapy comprises chemotherapy, radiation therapy, immunotherapy, a targeted therapy, or surgery.

In some embodiments, the method further comprises determining, identifying, or applying the value of the first or second phenotypic tumor heterogeneity score or the first or second composite tumor heterogeneity score for the sample as a diagnostic value associated with the sample.

In some embodiments, the method further comprises generating a genomic profile for the subject based on the determination of the first or second phenotypic tumor heterogeneity score or the first or second composite tumor heterogeneity score.

In some embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.

In some embodiments, the genomic profile for the subject further comprises results from a nucleic acid sequencing-based test.

In some embodiments, the method further comprises selecting an anti-cancer therapy, administering an anti-cancer therapy, or applying an anti-cancer therapy to the subject based on the generated genomic profile.

In some embodiments, the determination of the first or second phenotypic tumor heterogeneity score or the first or second composite tumor heterogeneity score for the sample is used in making suggested treatment decisions for the subject.

In some embodiments, the determination of the first or second phenotypic tumor heterogeneity score or the first or second composite tumor heterogeneity score for the sample is used in applying or administering a treatment to the subject.

An exemplary system comprises: a) one or more processors; and b) a memory communicatively coupled to the one or more processors and configured to store instructions that, when executed by the one or more processors, cause the system to: i) obtain a medical image associated with a sample from a subject; ii) identify a plurality of patches from a medical image; iii) identify a plurality of patch groups, wherein each patch group comprises one or more patches of the plurality of patches and corresponds to a region of interest in the medical image; iv) input each patch group into a trained machine learning model to generate a plurality of genomic alteration predictions corresponding to the plurality of patch groups, wherein the plurality of genomic alteration predictions is related to the presence of one or more genomic alterations in each of the input patch groups; and v) generate a phenotypic tumor heterogeneity score by comparing the plurality of genomic alteration predictions.

In some embodiments, the medical image comprises a histopathology image.

In some embodiments, the system further comprises instructions that, when executed by the one or more processors, cause the system to: obtain a genomic tumor heterogeneity score based on sequence read data associated with the sample; and calculate a composite tumor heterogeneity score based on the phenotypic tumor heterogeneity score and the genomic tumor heterogeneity score.

In some embodiments, the composite tumor heterogeneity score is a weighted average or a binarization of the phenotypic tumor heterogeneity score and the genomic tumor heterogeneity score.

In some embodiments, the system further comprises: identifying a combination of treatments for the subject based on the composite tumor heterogeneity score.

In some embodiments, the system further comprises: determining a monitoring plan for the subject based on the phenotypic tumor heterogeneity score before sequencing the sample from the subject.

In some embodiments, the system further comprises: if the composite tumor heterogeneity score is less than or equal to a threshold tumor heterogeneity score, predicting the subject to have a longer duration of therapeutic response to an anti-cancer therapy; and if the composite tumor heterogeneity score is more than the threshold tumor heterogeneity score, predicting the subject to have a shorter duration of therapeutic response to the anti-cancer therapy.

In some embodiments, the system further comprises: if the composite tumor heterogeneity score is less than or equal to the threshold tumor heterogeneity score, predicting the subject to have a higher survival rate if treated with an anti-cancer therapy; and if the composite tumor heterogeneity score is more than the threshold tumor heterogeneity score, predicting the subject to have a lower survival rate if treated with the anti-cancer therapy.

An exemplary system comprises: a) one or more processors; and b) a memory communicatively coupled to the one or more processors and configured to store instructions that, when executed by the one or more processors, cause the system to: i) obtain a medical image associated with a sample from a subject; ii) input the medical image into a trained machine learning model to identify a plurality of phenotypic features in the medical image; iii) identify a plurality of patches from the medical image; iv) identify a plurality of patch groups, wherein each patch group comprises one or more patches of the plurality of patches and corresponds to a region of interest in the medical image; v) obtain, based on the plurality of phenotypic features, a plurality of microenvironment profiles corresponding to the plurality of patch groups; and vi) generate a phenotypic tumor heterogeneity score by comparing the plurality of microenvironment profiles.

In some embodiments, the medical image comprises a histopathology image.

In some embodiments, the system further comprises instructions that, when executed by the one or more processors, cause the system to: obtain a genomic tumor heterogeneity score based on sequence read data associated with the sample from the subject; and calculate a composite tumor heterogeneity score based on the phenotypic tumor heterogeneity score and the genomic tumor heterogeneity score.

In some embodiments, the composite tumor heterogeneity score is a weighted average or a binarization of the phenotypic tumor heterogeneity score and the genomic tumor heterogeneity score.

In some embodiments, the system further comprises: identifying a combination of treatments for the subject based on the composite tumor heterogeneity score.

In some embodiments, the system further comprises: determining a monitoring plan for the subject based on the phenotypic tumor heterogeneity score before sequencing the sample from the subject.

In some embodiments, the system further comprises: if the composite tumor heterogeneity score is less than or equal to a threshold tumor heterogeneity score, predicting the subject to have a longer duration of therapeutic response to an anti-cancer therapy; and if the composite tumor heterogeneity score is more than the threshold tumor heterogeneity score, predicting the subject to have a shorter duration of therapeutic response to the anti-cancer therapy.

In some embodiments, the system further comprises: if the composite tumor heterogeneity score is less than or equal to the threshold tumor heterogeneity score, predicting the subject to have a higher survival rate if treated with an anti-cancer therapy; and if the composite tumor heterogeneity score is more than the threshold tumor heterogeneity score, predicting the subject to have a lower survival rate if treated with the anti-cancer therapy.

An exemplary system comprises: a) one or more processors; and b) a memory communicatively coupled to the one or more processors and configured to store instructions that, when executed by the one or more processors, cause the system to: i) obtain a medical image associated with a sample from a subject; ii) identify a plurality of patches from the medical image; iii) identify a plurality of patch groups, wherein each patch group comprises one or more patches of the plurality of patches and corresponds to a region of interest in the medical image; iv) input each patch group into a trained machine learning model to generate a plurality of embeddings corresponding to the plurality of patch groups; and v) generate a phenotypic tumor heterogeneity score by comparing the plurality of embeddings.

In some embodiments, the medical image comprises a histopathology image.

In some embodiments, the system further comprises instructions that, when executed by the one or more processors, cause the system to: obtain a genomic tumor heterogeneity score based on sequence read data associated with the sample from the subject; and calculate a composite tumor heterogeneity score based on the phenotypic tumor heterogeneity score and the genomic tumor heterogeneity score.

In some embodiments, the composite tumor heterogeneity score is a weighted average or a binarization of the phenotypic tumor heterogeneity score and the genomic tumor heterogeneity score.

In some embodiments, the system further comprises: identifying a combination of treatments for the subject based on the composite tumor heterogeneity score.

In some embodiments, the system further comprises: determining a monitoring plan for the subject based on the phenotypic tumor heterogeneity score before sequencing the sample from the subject.

In some embodiments, the system further comprises: if the composite tumor heterogeneity score is less than or equal to a threshold tumor heterogeneity score, predicting the subject to have a longer duration of therapeutic response to an anti-cancer therapy; and if the composite tumor heterogeneity score is more than the threshold tumor heterogeneity score, predicting the subject to have a shorter duration of therapeutic response to the anti-cancer therapy.

In some embodiments, the system further comprises: if the composite tumor heterogeneity score is less than or equal to the threshold tumor heterogeneity score, predicting the subject to have a higher survival rate if treated with an anti-cancer therapy; and if the composite tumor heterogeneity score is more than the threshold tumor heterogeneity score, predicting the subject to have a lower survival rate if treated with the anti-cancer therapy.

An exemplary non-transitory computer-readable storage medium stores one or more programs, the one or more programs comprising instructions, which when executed by one or more processors of a system, cause the system to: a) obtain a medical image associated with a sample from a subject; b) identify a plurality of patches from a medical image; c) identify a plurality of patch groups, wherein each patch group comprises one or more patches of the plurality of patches and corresponds to a region of interest in the medical image; d) input each patch group into a trained machine learning model to generate a plurality of genomic alteration predictions corresponding to the plurality of patch groups, wherein the plurality of genomic alteration predictions is related to the presence of one or more genomic alterations in each of the input patch groups; and e) generate a phenotypic tumor heterogeneity score by comparing the plurality of genomic alteration predictions.

In some embodiments, the medical image comprises a histopathology image.

In some embodiments, the non-transitory computer-readable storage medium further comprises instructions that, when executed by one or more processors of a system, cause the system to: obtain a genomic tumor heterogeneity score based on sequence read data associated with the sample; and calculate a composite tumor heterogeneity score based on the phenotypic tumor heterogeneity score and the genomic tumor heterogeneity score.

In some embodiments, the composite tumor heterogeneity score is a weighted average or a binarization of the phenotypic tumor heterogeneity score and the genomic tumor heterogeneity score.

In some embodiments, the non-transitory computer-readable storage medium further comprises: identifying a combination of treatments for the subject based on the composite tumor heterogeneity score.

In some embodiments, the non-transitory computer-readable storage medium further comprises: determining a monitoring plan for the subject based on the phenotypic tumor heterogeneity score before sequencing the sample from the subject.

In some embodiments, the non-transitory computer-readable storage medium further comprises: if the composite tumor heterogeneity score is less than or equal to a threshold tumor heterogeneity score, predicting the subject to have a longer duration of therapeutic response to an anti-cancer therapy; and if the composite tumor heterogeneity score is more than the threshold tumor heterogeneity score, predicting the subject to have a shorter duration of therapeutic response to the anti-cancer therapy.

In some embodiments, the non-transitory computer-readable storage medium further comprises: if the composite tumor heterogeneity score is less than or equal to the threshold tumor heterogeneity score, predicting the subject to have a higher survival rate if treated with an anti-cancer therapy; and if the composite tumor heterogeneity score is more than the threshold tumor heterogeneity score, predicting the subject to have a lower survival rate if treated with the anti-cancer therapy.

An exemplary non-transitory computer-readable storage medium stores one or more programs, the one or more programs comprising instructions, which when executed by one or more processors of a system, cause the system to: a) obtain a medical image associated with a sample from a subject; b) input the medical image into a trained machine learning model to identify a plurality of phenotypic features in the medical image; c) identify a plurality of patches from the medical image; d) identify a plurality of patch groups, wherein each patch group comprises one or more patches of the plurality of patches and corresponds to a region of interest in the medical image; e) obtain, based on the plurality of phenotypic features, a plurality of microenvironment profiles corresponding to the plurality of patch groups; and f) generate a phenotypic tumor heterogeneity score by comparing the plurality of microenvironment profiles.

In some embodiments, the medical image comprises a histopathology image.

In some embodiments, the non-transitory computer-readable storage medium further comprises instructions that, when executed by one or more processors of a system, cause the system to: obtain a genomic tumor heterogeneity score based on sequence read data associated with the sample from the subject; and calculate a composite tumor heterogeneity score based on the phenotypic tumor heterogeneity score and the genomic tumor heterogeneity score.

In some embodiments, the composite tumor heterogeneity score is a weighted average or a binarization of the phenotypic tumor heterogeneity score and the genomic tumor heterogeneity score.

In some embodiments, the non-transitory computer-readable storage medium further comprises: identifying a combination of treatments for the subject based on the composite tumor heterogeneity score.

In some embodiments, the non-transitory computer-readable storage medium further comprises: determining a monitoring plan for the subject based on the phenotypic tumor heterogeneity score before sequencing the sample from the subject.

In some embodiments, the non-transitory computer-readable storage medium further comprises: if the composite tumor heterogeneity score is less than or equal to a threshold tumor heterogeneity score, predicting the subject to have a longer duration of therapeutic response to an anti-cancer therapy; and if the composite tumor heterogeneity score is more than the threshold tumor heterogeneity score, predicting the subject to have a shorter duration of therapeutic response to the anti-cancer therapy.

In some embodiments, the non-transitory computer-readable storage medium further comprises: if the composite tumor heterogeneity score is less than or equal to the threshold tumor heterogeneity score, predicting the subject to have a higher survival rate if treated with an anti-cancer therapy; and if the composite tumor heterogeneity score is more than the threshold tumor heterogeneity score, predicting the subject to have a lower survival rate if treated with the anti-cancer therapy.

An exemplary non-transitory computer-readable storage medium stores one or more programs, the one or more programs comprising instructions, which when executed by one or more processors of a system, cause the system to: a) obtain a medical image associated with a sample from a subject; b) identify a plurality of patches from the medical image; c) identify a plurality of patch groups, wherein each patch group comprises one or more patches of the plurality of patches and corresponds to a region of interest in the medical image; d) input each patch group into a trained machine learning model to generate a plurality of embeddings corresponding to the plurality of patch groups; and e) generate a phenotypic tumor heterogeneity score by comparing the plurality of embeddings.

In some embodiments, the medical image comprises a histopathology image.

In some embodiments, the non-transitory computer-readable storage medium further comprises instructions that, when executed by one or more processors of a system, cause the system to: obtain a genomic tumor heterogeneity score based on sequence read data associated with the sample from the subject; and calculate a composite tumor heterogeneity score based on the phenotypic tumor heterogeneity score and the genomic tumor heterogeneity score.

In some embodiments, the composite tumor heterogeneity score is a weighted average or a binarization of the phenotypic tumor heterogeneity score and the genomic tumor heterogeneity score.

In some embodiments, the non-transitory computer-readable storage medium further comprises: identifying a combination of treatments for the subject based on the composite tumor heterogeneity score.

In some embodiments, the non-transitory computer-readable storage medium further comprises: determining a monitoring plan for the subject based on the phenotypic tumor heterogeneity score before sequencing the sample from the subject.

In some embodiments, the non-transitory computer-readable storage medium further comprises: if the composite tumor heterogeneity score is less than or equal to a threshold tumor heterogeneity score, predicting the subject to have a longer duration of therapeutic response to an anti-cancer therapy; and if the composite tumor heterogeneity score is more than the threshold tumor heterogeneity score, predicting the subject to have a shorter duration of therapeutic response to the anti-cancer therapy.

In some embodiments, the non-transitory computer-readable storage medium further comprises: if the composite tumor heterogeneity score is less than or equal to the threshold tumor heterogeneity score, predicting the subject to have a higher survival rate if treated with an anti-cancer therapy; and if the composite tumor heterogeneity score is more than the threshold tumor heterogeneity score, predicting the subject to have a lower survival rate if treated with the anti-cancer therapy.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process for determining a phenotypic tumor heterogeneity score in a sample from an individual, in accordance with one embodiment of the present disclosure.

FIG. 2A provides a non-limiting example of a process for identifying a plurality of patches in an image, in accordance with one embodiment of the present disclosure.

FIG. 2B provides three non-limiting examples of approaches to identify a plurality of patch groups corresponding to a plurality of regions of interest in an image, in accordance with one embodiment of the present disclosure.

FIG. 3A provides a non-limiting example of the utilization of a single-mutation prediction model to calculate a phenotypic tumor heterogeneity score, in accordance with one embodiment of the present disclosure.

FIG. 3B provides a non-limiting example of the utilization of a single-mutation prediction model to calculate a phenotypic tumor heterogeneity score, in accordance with one embodiment of the present disclosure.

FIG. 4A provides a non-limiting example of the utilization of a multiple-mutation prediction model to calculate a phenotypic tumor heterogeneity score, in accordance with one embodiment of the present disclosure.

FIG. 4B provides a non-limiting example of the utilization of a multiple-mutation prediction model to calculate a phenotypic tumor heterogeneity score, in accordance with one embodiment of the present disclosure.

FIG. 5 provides a non-limiting example of a process for determining a phenotypic tumor heterogeneity score in a sample from an individual, in accordance with one embodiment of the present disclosure.

FIG. 6 provides a non-limiting example of a process for determining phenotypic tumor heterogeneity based on tumor microenvironment profiles, in accordance with one embodiment of the present disclosure.

FIG. 7 provides a non-limiting example of a process for determining phenotypic tumor heterogeneity score in a sample from an individual, in accordance with one embodiment of the present disclosure.

FIG. 8 provides a non-limiting example of a process for determining phenotypic tumor heterogeneity based on embeddings, in accordance with one embodiment of the present disclosure.

FIG. 9 depicts an exemplary computing device or system in accordance with one embodiment of the present disclosure.

FIG. 10 depicts an exemplary computer system or computer network, in accordance with some instances of the systems described herein.

### DETAILED DESCRIPTION

Methods and systems for evaluating tumor heterogeneity using histopathology images are described. Some embodiments of the present disclosure can determine a phenotypic/morphological tumor heterogeneity score associated a tumor sample by comparing the mutation status of one or more genomic alterations across multiple regions of interest in a histopathology image of the tumor sample. An exemplary system can obtain an image (e.g., a medical image) associated with a sample from an individual. The system can identify a plurality of patches from the medical image and then identify a plurality of patch groups, each patch group comprising one or more patches of the plurality of patches and corresponds to a region of interest in the medical image. The system can input each patch group into a trained machine learning model to generate a plurality of genomic alteration predictions corresponding to the plurality of patch groups. The plurality of genomic alteration predictions includes the presence of one or more genomic alterations in each of the input patch groups. The system can then generate a phenotypic tumor heterogeneity score by comparing the plurality of genomic alteration predictions. In some embodiments, the model is configured to predict the mutation status of a single alteration (e.g., EGFR only), and the system can then generate an alteration-specific clonality estimate and a subsequent heterogeneity score. In some embodiments, the model is configured to predict the mutational statuses for a multitude of genes rather than a single one.

Some embodiments of the present disclosure can determine a phenotypic tumor heterogeneity score associated a tumor sample by comparing a tumor microenvironment profile across multiple regions of interest in a histopathology image of the tumor sample. An exemplary system can obtain a medical image associated with the sample from the individual and input the medical image into a trained machine learning model to identify a plurality of phenotypic features in the medical image. The system can identify a plurality of patches from the medical image and then identify a plurality of patch groups. Each patch group comprises one or more patches of the plurality of patches and corresponds to a region of interest in the medical image. The system can obtain, based on the plurality of phenotypic features, a plurality of microenvironment profiles corresponding to the plurality of patch groups and generate a phenotypic tumor heterogeneity score by comparing the plurality of microenvironment profiles.

Some embodiments of the present disclosure can determine a phenotypic tumor heterogeneity score associated a tumor sample by comparing numerical representations (e.g., embeddings) corresponding to multiple regions of interest in a histopathology image of the tumor sample. An exemplary system can obtain an image (e.g., a medical image) associated with a sample from an individual. The system can identify a plurality of patches from the medical image and then identify a plurality of patch groups, each patch group comprising one or more patches of the plurality of patches and corresponds to a region of interest in the medical image. The system can input each patch group into a trained machine learning model to generate a plurality of embeddings corresponding to the plurality of patch groups and generate a phenotypic tumor heterogeneity score by comparing the plurality of embeddings.

Techniques described herein may be used to analyze a sample such as a tissue biopsy sample, a liquid biopsy sample, or a normal control. For example, some embodiments of the present disclosure may be used to analyze a blood smear slide to examine free-floating tumor-cell heterogeneity (e.g., at a cellular level).

In any of the embodiments above, the phenotypic tumor heterogeneity score can be combined with a genomic tumor heterogeneity score to generate a composite tumor heterogeneity score. In some embodiments, the composite score can provide a view on tumor heterogeneity that uses both bulk sequencing but also captures orthogonal information from imaging that may provide spatial insight in a way that may be difficult with bulk sequencing derived comprehensive genomic profiling (CGP). In some embodiments, the composite score can be optimized and tuned for effectiveness at stratifying patient populations on outcomes. For example, the system can identify one or more threshold composite scores for stratifying patients based on the outcome data (e.g., response to one or more treatments, survival, relapse, etc.).

The genomic tumor heterogeneity score may be obtained from sequencing data associated with the same sample depicted in the image (e.g., whole slide image), a different sample of the subject, and/or a sample from a comparable subject. In some embodiments, the genomic tumor heterogeneity score comprises a bulk sequencing genomics-based tumor heterogeneity score that uses a cancer cell fraction (CCF) estimation. CCF may be defined as the fraction of tumor cells from the sequenced specimen carrying a set of short variants. In some embodiments, the CCF of every short variant (SV) detected in the specimen is estimated. The estimated CCFs can be used to generate the genomic heterogeneity score, which may be calculated as the ratio of the median CCF of all short variants to the quartile coefficient of dispersion (QCD) of all short variants. In some embodiments, the genomic tumor heterogeneity score is calculated using data derived from a tissue specimen's single region bulk tumor targeted exome assay. Additional details of exemplary genomic tumor heterogeneity score can be found in, for example, PCT Application No. PCT/US2023/064598, titled “CHARACTERIZATION OF TUMOR HETEROGENEITY AS A PROGNOSTIC BIOMARKER,” the content of which is incorporated herein by reference.

Measuring the heterogeneity of tumors based on a single, pre-treatment tissue biopsy according to some embodiments in the present disclosure may give insight into the duration of therapeutic response based on Darwinian evolution principles of tumors under a given selection pressure. For example, tumors with little genomic diversity and from an evolutionary standpoint having lesser overall fitness may require more time to accumulate the necessary mutations required to achieve resistance. In contrast, tumors with higher genomic diversity may require less time to accumulate and select for a resistance mutation and therefore associate with shorter response durations. In this way, measuring the baseline tumor's heterogeneity may serve to predict the duration of response to a particular therapy.

Accordingly, understanding tumor heterogeneity using embodiments of the present disclosure can allow development of prognostic biomarkers to provide insight to duration of therapeutic response (e.g., at tissue baseline) and development of combinatorial treatment options, as well as providing opportunity to recommend longitudinal monitoring based clinical interventions. For example, NSCLC patients who progress on Osimertinib (Third generation TKI) due to acquired resistance from a MET amplification can be treated with a combination of Osimertinib and MET inhibitor. As another example, patients with a TP53 mutation may be treated with Osimertinib plus Carboplatin/Etoposide; patients with RAS/RAF/MAPK alterations may be treated with Osimertinib plus a MEK inhibitor; and patients EGFR C797 alterations may be treated with Osimertinib plus a fourth-generation EGFR TKI. Predicting the duration of therapeutic benefit at baseline can have significant clinical implications for combinatorial treatment strategies, next-line therapy decisions, and tailoring novel monitoring capabilities based on likelihood of relapse.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The section headings used herein are for organizational purposes only and are not to be construed as limiting the subject matter described.

### Methods for Determining Tumor Heterogeneity Based on Genomic Alteration Predictions

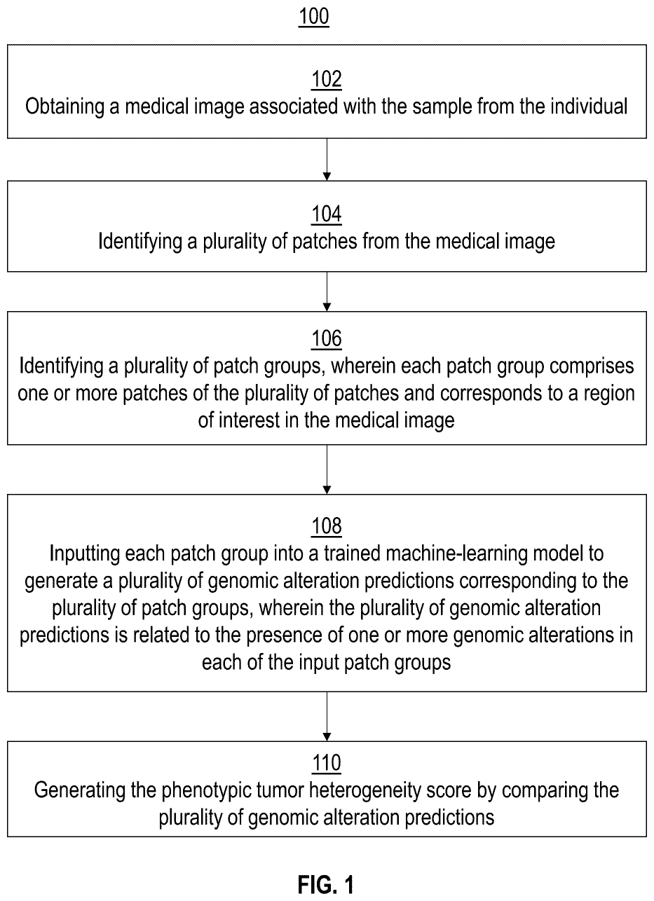

FIG. 1 illustrates an exemplary process for determining a phenotypic tumor heterogeneity score in a sample from an individual, in accordance with some embodiments. Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At block 102, an exemplary system (e.g., one or more electronic devices) obtains an image associated with the sample from the individual. In some embodiments, the image is a medical image. In some examples, the image comprises a histopathology image such as an immunohistochemistry (IHC) stain image, a hematoxylin and eosin (H&E) stain image, or any combination thereof.

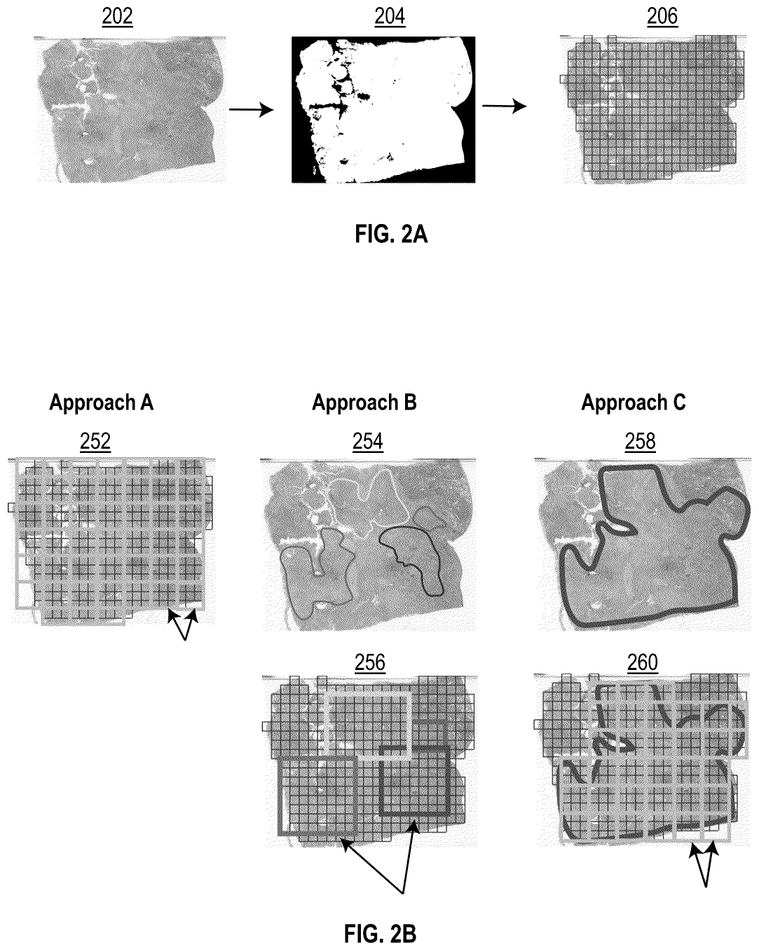

At block 104, the system identifies a plurality of patches from the medical image. In some embodiments, the system may first identify a tumor tissue depicted in the medical image, for example, by inputting the medical image into a trained segmentation machine learning model. The system may then divide the portion of the image depicting the tumor tissue into a plurality of patches. Further exemplary details for identifying the plurality of patches in the medical image are provided herein with reference to FIG. 2A.

At block 106, the system identifies a plurality of patch groups corresponding to a plurality of regions of interest. Each patch group comprises one or more patches of the plurality of patches and corresponds to a region of interest in the medical image. In some embodiments, the plurality of regions of interest may correspond to different visually and/or morphologically distinct portions of a tumor tissue in the medical image. In some embodiments, the plurality of regions of interest may correspond to different local neighborhoods of the tumor tissue in the medical image. In some embodiments, the plurality of regions of interest may correspond to the individual patches. Exemplary methods for identifying the plurality of regions of interest are provided herein with reference to FIG. 2B.

At block 108, the system inputs each patch group into a trained machine learning model to generate a plurality of genomic alteration predictions corresponding to the plurality of patch groups. The plurality of genomic alteration predictions includes the presence of one or more genomic alterations in each of the input patch groups.

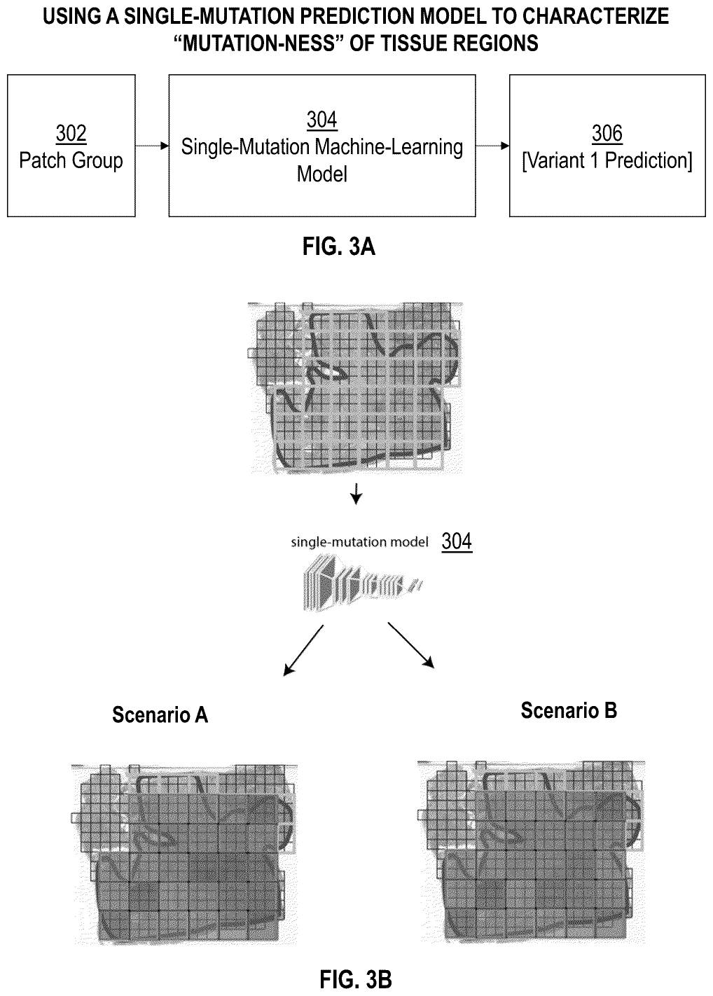

In some embodiments, the trained machine learning model is configured to receive an input patch group and output a single prediction of the presence of a single genomic alteration in the patch group. The single genomic alteration may be: an epidermal growth factor receptor (EGFR) driver mutation. a BRAF driver mutation, a MET driver mutation, an ERBB2 (HER2) driver mutation, a PIK3CA driver mutation, a KRAS driver mutation, an NRAS driver mutation, a BRCA1 driver mutation, a BRCA2 driver mutation, an ATM driver mutation, an FGFR2 driver mutation, a driver mutation in a homologous recombination repair (HRR) gene, an NTRK1 driver mutation, an NTRK2 driver mutation, an NTRK3 driver mutation, an RET driver mutation, an ALK mutation, or a ROS1 mutation. Additional details of the single-mutation prediction model are provided herein with reference to FIGS. 3A-B.

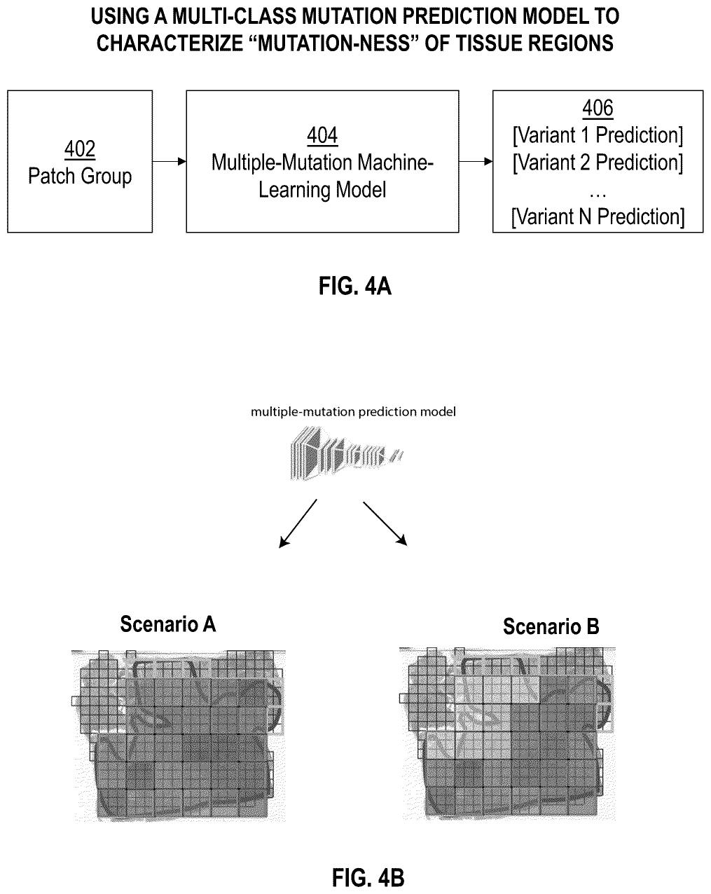

In some embodiments, the trained machine learning model is configured to receive an input patch group and output a plurality of predictions of the presence of a plurality of genomic alterations in the patch group. In some embodiments, the plurality of predictions is configured to be dependent on each other. For example, the sum of the plurality of predictions can be configured to be a fixed value (e.g., 1). In some embodiments, the plurality of predictions is independent of each other. The plurality of genomic alterations may comprise: an epidermal growth factor receptor (EGFR) driver mutation. a BRAF driver mutation, a MET driver mutation, an ERBB2 (HER2) driver mutation, a PIK3CA driver mutation, a KRAS driver mutation, an NRAS driver mutation, a BRCA1 driver mutation, a BRCA2 driver mutation, an ATM driver mutation, an FGFR2 driver mutation, a driver mutation in a homologous recombination repair (HRR) gene, an NTRK1 driver mutation, an NTRK2 driver mutation, an NTRK3 driver mutation, an RET driver mutation, an ALK mutation, a ROS1 mutation, or any combination thereof. Additional details of the multiple-mutation prediction model are provided herein with reference to FIGS. 4A-B.

As examples, for a variety of oncogenic driver mutations in LUAD cancers (e.g., KRAS, EGFR), there are strong observed associations of certain histological subtypes. EGFR mutations in LUAD are positively associated with lepidic and acinar histological subtypes, as well as having negative association with high levels of immune infiltration. These features are detectable from the histopathology whole slide image. Thus, the trained machine learning model can provide mutation predictions with particularly higher performance.

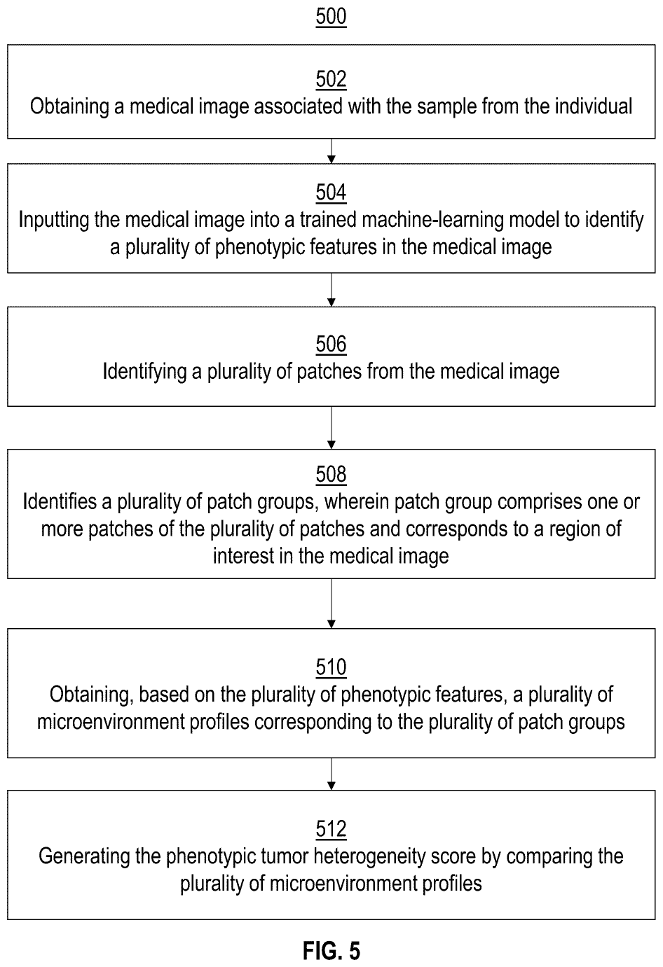

The machine learning model may comprise a convolutional neural network model and/or a vision transformer model configured to receive image data (e.g., raw pixel inputs). In some embodiments, the convolutional neural network model comprises an attention mechanism. The attention mechanisms can treat biomarker/alteration prediction problems as a multiple-instance formulation, where a patch group is evaluated together rather than patch-by-patch. Similarly, a vision transformer model can evaluate a patch group together rather than patch-by-patch, with an added component of explicit spatial and sequence information provided to the model for each patch. In some embodiments, the machine learning model may comprise a decision tree model, a random forest model, and/or a gradient boosting model. The model may be configured to receive generated features corresponding to a patch group, such as cell characteristics as described herein (e.g., with reference to FIG. 5). In some embodiments, each patch group may be configured to comprise a single patch and the machine learning model may be a model configured to receive a single patch and provide a patch-level prediction.

At block 110, the system generates the phenotypic tumor heterogeneity score by comparing the plurality of genomic alteration predictions. As discussed above with reference to block 108, in some embodiments, the trained machine learning model is configured to receive an input patch group and output a single prediction of the presence of a single genomic alteration in the patch group. Accordingly, the system can assign the phenotypic tumor heterogeneity score to a first value indicative of lower tumor heterogeneity if the plurality of genomic alteration predictions all indicates the presence of the single genomic alteration, and assign the phenotypic tumor heterogeneity score to a second value indicative of higher tumor heterogeneity if some of the plurality of genomic alteration predictions indicate the presence of the single genomic alteration and some of the plurality of genomic alteration predictions do not indicate the presence of the single genomic alteration.

Alternatively, the system can calculate a variance value based on the plurality of genomic alteration predictions, assign the phenotypic tumor heterogeneity score to a first value indicative of lower tumor heterogeneity if the variance value is lower than or equal to a predefined threshold, and assign the phenotypic tumor heterogeneity score to a second value indicative of higher tumor heterogeneity if the variance value is higher than a predefined threshold. In some embodiments, the phenotypic tumor heterogeneity score is proportional to the variance value.

As discussed above with reference to block 108, in some embodiments, the trained machine learning model is configured to receive an input patch group and output a plurality of predictions of the presence of a plurality of genomic alterations in the patch group. Accordingly, the system can generate a plurality of phenotypic tumor heterogeneity sub-scores corresponding to the plurality of genomic alterations, and calculate the phenotypic tumor heterogeneity score based on the plurality of phenotypic tumor heterogeneity sub-scores (e.g., by averaging). Each sub-score may be, for example, a value indicative of the variance/spread among predictions with respect to a singe genomic alteration.

The phenotypic tumor heterogeneity score can be combined with a genomic tumor heterogeneity score to generate a composite tumor heterogeneity score. The genomic tumor heterogeneity score may be obtained from sequencing data associated with the same sample depicted in the image and/or a different sample of the subject. The composite tumor heterogeneity score can be calculated based on the phenotypic tumor heterogeneity score and the genomic tumor heterogeneity score. In some embodiments, the composite tumor heterogeneity score may be a weighted average of the phenotypic tumor heterogeneity score and the genomic tumor heterogeneity score. In some embodiments, the composite tumor heterogeneity score may be a composite binarization or a function of a categorical phenotypic tumor heterogeneity score and a categorical genomic tumor heterogeneity score. Alternatively, the composite tumor heterogeneity score may be a binarization of the phenotypic tumor heterogeneity score and the genomic tumor heterogeneity score. For example, if both the genomic tumor heterogeneity score and the phenotypic tumor heterogeneity score are indicative of high heterogeneity, then the system can assign the composite tumor heterogeneity score to be indicative of high heterogeneity.

The composite tumor heterogeneity score can be used for a number of downstream applications. In some embodiments, the composite tumor heterogeneity score can be used to identify a combination of treatments for the individual. For example, if the composite tumor heterogeneity score is indicative of high heterogeneity, the system may determine a combination of treatment for the individual. As another example, if the composite tumor heterogeneity score is indicative of high heterogeneity, the system may provide a recommendation to monitor the subject and/or the subject's response to treatments earlier and/or more frequently.

In some embodiments, the composite tumor heterogeneity score can be used to predict the duration of a therapeutic response to the anti-cancer therapy. For example, if the composite tumor heterogeneity score is less than or equal to a threshold tumor heterogeneity score, the system may predict the individual to have a longer duration of therapeutic response to an anti-cancer therapy. On the other hand, if the composite tumor heterogeneity score is more than the threshold tumor heterogeneity score, the system may the individual to have a shorter duration of therapeutic response to the anti-cancer therapy.

In some embodiments, the composite tumor heterogeneity score can be used to predict the survival rate of the subject if treated with an anti-cancer therapy. For example, if the composite tumor heterogeneity score is less than or equal to the threshold tumor heterogeneity score, the system may predict the individual to have a higher survival rate if treated with an anti-cancer therapy. On the other hand, if the composite tumor heterogeneity score is more than the threshold tumor heterogeneity score, the system may predict the individual to have a lower survival rate if treated with the anti-cancer therapy.

In some embodiments, the phenotypic tumor heterogeneity score may be determined before the genomic tumor heterogeneity score is determined. This may be because the sequencing data of the subject, which is needed to calculate the genomic tumor heterogeneity score, may take longer time to obtain than histopathology images of the subject. Accordingly, the phenotypic tumor heterogeneity score may be used for early decision-making before samples of the subject are sequenced and/or before the genomic tumor heterogeneity score is available. The phenotypic tumor heterogeneity score may be used for any of the downstream applications described above by itself. For example, the system can determine a monitoring plan for the individual based on the phenotypic tumor heterogeneity score before sequencing the sample from the individual.

FIG. 2A illustrates an exemplary process for identifying a plurality of patches in an image, in accordance with some embodiments. With reference to FIG. 2A, an exemplary system can receive a histopathology whole slide image 202, which has a breadth of morphologies that are spatially distributed. The system can analyze the image 202 to obtain a pixel-wise segmentation mask 204, which can indicate, for each pixel in the image 202, whether the pixel depicts a tumor tissue or not. The pixel-wise segmentation mask 204 may be identified, for example, by inputting the image 202 into a trained machine learning model configured to identify tumor tissues in the input image and output a segmentation mask. Based on the segmentation mask 204, the system can then divide the portion of the image depicting tumor tissue(s) into a plurality of patches, as shown by the grids in image 206. The plurality of patches can be then provided to various machine learning models for further analyses, as described herein. Dividing an image into a plurality of patches can be technically advantageous because the original images, such as histopathology whole slide images, are usually very large. Training a machine learning model to process image patches, rather than the whole image, can allow the models to be trained more efficiently and accurately and allow the models to perform more efficiently and accurately during runtime when processing new image data.

FIG. 2B illustrates three exemplary approaches to identify a plurality of patch groups corresponding to a plurality of regions of interest in an image, in accordance with some embodiments. In Approach A, the system can systematically partition out an image or a portion thereof into local neighborhoods. Each neighborhood is a region of interest and contains one or more patches. In the depicted example 252, the local neighborhood (as indicated by larger, green grids) correspond to adjacent groups of patches and each local neighborhood contains at least one but no more than 9 patches (as indicated by smaller, black grids). The patches within the same neighborhood (i.e., the same region of interest) can be inputted into a model (e.g., as a group, patch by patch, a randomly sampled subset of patches from the group) to obtain a single output corresponding to the neighborhood, as described herein.

In Approach B, the system determines geographically separate regions of interest. In the depicted example 254, the system identifies four geographically separate regions of interest. Each region of interest corresponds to an area in the image that depicts tissue of interest. In some embodiments, the four regions may be identified based on annotations of a pathologist (e.g., based on their visual or morphological characteristics). In some embodiments, the four regions may be identified by inputting the image into a machine learning model configured to identify one or more regions of interest in the image (e.g., visually distinct regions, morphologically distinct regions). These may correspond to, for example, separate tumor masses or swathes of tumor belonging to the same mass. Based on the regions of interest identified in image 254, the system can identify a plurality of patch groups corresponding to those regions, as indicated in the four patch groups in example 256. The patches within the same patch group (i.e., the same region of interest) can be inputted into a model (e.g., as a group, patch by patch, a randomly sampled subset of patches from the group) to obtain a single output corresponding to the neighborhood, as described herein.

In Approach C, the system first determines a large continuous area (e.g., corresponding to the tumor mass) and then create local neighborhoods based on the large continuous area. In the depicted example 258, the system first identifies a single continuous area (e.g., based on a machine learning model) and then systematically partitions out the local neighborhoods within the continuous area. The patches within the same neighborhood (i.e., the same region of interest) can be inputted into a model (e.g., as a group, patch by patch, a randomly sampled subset of patches from the group) to obtain a single output corresponding to the neighborhood, as described herein. It should be appreciated that the approaches described with reference to FIG. 2B are merely exemplary and can be combined in any manner. In some embodiments, a region of interest and/or a patch group may comprise a single patch.

FIGS. 3A-B illustrate the utilization of a single-mutation prediction model to calculate a phenotypic tumor heterogeneity score, in accordance with some embodiments. With reference to FIG. 3A, the trained machine learning model 304 is configured to receive an input patch group 302 and output a single prediction 306 of the presence of a single genomic alteration in the patch group (e.g., the presence/absence of Variant 1). The single prediction may be a binary value (e.g., indicative of presence/absence), a continuous value (e.g., indicative of a probability of presence), an integer (e.g., high, medium, low presence). The single genomic alteration may be: an epidermal growth factor receptor (EGFR) driver mutation. a BRAF driver mutation, a MET driver mutation, an ERBB2 (HER2) driver mutation, a PIK3CA driver mutation, a KRAS driver mutation, an NRAS driver mutation, a BRCA1 driver mutation, a BRCA2 driver mutation, an ATM driver mutation, an FGFR2 driver mutation, a driver mutation in a homologous recombination repair (HRR) gene, an NTRK1 driver mutation, an NTRK2 driver mutation, an NTRK3 driver mutation, an RET driver mutation, an ALK mutation, or a ROS1 mutation.

With reference to FIG. 3B, each patch group (as indicated by the green grids) of an image is inputted into single-mutation model 304 to obtain a prediction of the presence/absence of a given genomic mutation in the patch group. In Scenario A, all patch groups have strong predictions for the give mutation, showing low heterogeneity. Accordingly, in Scenario A, the system can assign the phenotypic tumor heterogeneity score to a first value indicative of lower tumor heterogeneity. In Scenario B, some patch groups have strong predictions for the given mutation but other neighborhoods have low mutation predictions for the given mutation. Accordingly, in Scenario B, the system can assign the phenotypic tumor heterogeneity score to a second value indicative of higher tumor heterogeneity.

The system can quantify the difference among the predictions across different patch groups in an image using a number of metrics. In some embodiments, the system can calculate a continuous score quantifying the spread of the predictions (e.g., variance, standard deviation) across different patch groups. The continuous score can then be used to calculate the phenotypic tumor heterogeneity score. For example, the system can then assign the phenotypic tumor heterogeneity score to a first value indicative of lower tumor heterogeneity if the score is lower than or equal to a predefined threshold, and assign the phenotypic tumor heterogeneity score to a second value indicative of higher tumor heterogeneity if the score is higher than a predefined threshold. As another example, the phenotypic tumor heterogeneity score can be proportional to the continuous score.

In some embodiments, the metric is a binary value indicative of whether the predictions are all identical or not. The system can then assign the phenotypic tumor heterogeneity score to a first value indicative of lower tumor heterogeneity if the binary value indicates that all predictions are identical, and assign the phenotypic tumor heterogeneity score to a second value indicative of higher tumor heterogeneity if the binary value indicates that not all predictions are identical.

In one exemplary implementation, the trained machine learning model can be used to analyze a plurality of patch groups from an image of a non-small cell lung cancer (NSCLC) specimen, where EGFR driver alteration is typically considered to be truncal. If there are no regions that are predicted to be EGFR mutant, the specimen can be determined to be EGFR wild-type. If all sampled regions are predicted to be EGFR mutant, the EGFR mutation may be clonal and the specimen may be assigned a low phenotypic tumor heterogeneity score. Instead, if there are some regions that are predicted to be highly EGFR and other geographically separated regions are not predicted to be highly EGFR, then it may be possible that there are subclones within the cancer that harbor co-occurring mutations in addition to the clonal EGFR mutation that then affects the visually discernible features for tumor cells belonging to that subclones (e.g., histology, tumor microenvironment). Accordingly, the specimen can be assigned a high phenotypic tumor heterogeneity score. In this approach, the output of the trained machine learning model may not be a strict EGFR mutation prediction but rather an assessment of a region's EGFR-ness.

FIGS. 4A-B illustrate the utilization of a multiple-mutation prediction model to calculate a phenotypic tumor heterogeneity score, in accordance with some embodiments. With reference to FIG. 4A, the trained machine learning model 404 is configured to receive an input patch group 402 and output a plurality of predictions 406 of the presence of a plurality of genomic alterations in the patch group (e.g., presence/absence of Variant 1, presence/absence of Variant 2, . . . , presence/absence of Variant N). Each prediction may be a binary value (e.g., indicative of presence/absence) or a continuous value (e.g., indicative of a probability of presence). The plurality of genomic alterations may comprise: an epidermal growth factor receptor (EGFR) driver mutation. a BRAF driver mutation, a MET driver mutation, an ERBB2 (HER2) driver mutation, a PIK3CA driver mutation, a KRAS driver mutation, an NRAS driver mutation, a BRCA1 driver mutation, a BRCA2 driver mutation, an ATM driver mutation, an FGFR2 driver mutation, a driver mutation in a homologous recombination repair (HRR) gene, an NTRK1 driver mutation, an NTRK2 driver mutation, an NTRK3 driver mutation, an RET driver mutation, an ALK mutation, a ROS1 mutation, or any combination thereof. The collection of N predictions is referred to as a prediction profile herein.

In some embodiments, the plurality of predictions is configured to be dependent on each other. In some embodiments, the sum of the plurality of predictions can be configured to be a fixed value (e.g., 1). For example, the machine learning model 404 can be configured to output N predictions for N gene mutations, where each prediction must be constrained to the range [0,1] and all predictions must sum together to 1 (e.g., using a softmax output activation). This configuration may assume that multiple mutations that co-occur in the same localized tumor region cannot be fully expressed phenotypically in the same way that can be expressed if there was only one relevant mutation that occurred; instead, the model is configured to either make a highly confident prediction for only one of the N alterations, make a mixed prediction for a small selection of mutations within the N, or make a unconfident prediction that is diffused across all N choices. In other words, the value of each prediction of the N predictions can be indicative of the confidence of the respective prediction.

In some embodiments, the plurality of predictions is independent of each other. For example, the machine learning model 404 can be configured to output N predictions for N gene mutations, each prediction is constrained between [0,1] but there is no imposition that all predictions for the N alterations must sum to a fixed value (e.g., each alteration output's activation function is a separate sigmoid). For example, the model may provide a full confidence prediction that there is an EGFR alteration and also full confidence prediction that there is a TP53 co-mutation. In either multi-alteration prediction approach (whether each alteration prediction is dependent or independent of each other), tissue regions can directly be analyzed for co-occurring mutations. On a given slide, there might be a tumor region where the model only predicts EGFR mutation and another tumor region where the model predicts both EGFR and PIK3CA alterations, as discussed below.

With reference to FIG. 4B, each patch group (as indicated by the green grids in image 304 of FIG. 3B) is inputted into the machine learning model 404 to obtain a plurality of predictions of a plurality of genomic mutations in the patch group. In Scenario A, all patch groups have similar mutation prediction profiles, showing low heterogeneity. For example, all patch groups may have a mutation prediction profile of EGFR only. Accordingly, in Scenario A, the system can assign the phenotypic tumor heterogeneity score to a first value indicative of lower tumor heterogeneity. In Scenario B, the patch groups have different mutation prediction profiles. For example, one profile may indicate EGFR only, one profile may indicate EGFR+PIK3CA, and one profile may indicate EGFR+CTNNB1. Accordingly, in Scenario B, the system can assign the phenotypic tumor heterogeneity score to a second value indicative of higher tumor heterogeneity.

The system can quantify the difference among the prediction profiles across different patch groups in an image in a number of metrics. In some embodiments, for each mutation of the N mutations, the system can calculate a continuous score quantifying the spread of the predictions (e.g., variance, standard deviation) across different patch groups to obtain a mutation-specific sub-score. The sub-score can be then aggregated (e.g., averaged, summed) to obtain an overall score, which can then be used to obtain the phenotypic tumor heterogeneity score. For example, the phenotypic tumor heterogeneity score can be proportional to the overall score. For example, the system can then assign the phenotypic tumor heterogeneity score to a first value indicative of lower tumor heterogeneity if the overall score is lower than or equal to a predefined threshold, and assign the phenotypic tumor heterogeneity score to a second value indicative of higher tumor heterogeneity if the overall score is higher than a predefined threshold. In some embodiments, the metric is a binary value indicative of whether, for each mutation of the N mutations, the predictions are all identical or not across patch groups. The system can then assign the phenotypic tumor heterogeneity score by examining whether/how many of the N binary values indicate heterogeneity.

### Methods for Determining Tumor Heterogeneity Based on Microenvironment Profiles

From a model interpretability perspective, it may be desirable to train a model or a suite of models that can quantify and characterize the tumor microenvironment in addition to or instead of predicting biomarkers (e.g., gene alterations). For example, certain alterations have been observed to correlate with changes at the cellular level; tumor cells driven by these mutations may show notable differences in tumor cell shapes, cytoplasmic textures, and nuclei characteristics. An exemplary system can train end-to-end computer vision models to predict, classify, and/or segment relevant and specific phenotypic features. For example, a convolutional neural network may be trained to segment tumor cells, which would then allow tumor cell features such as polarity, concavity, texture, shape, and size to be subsequently calculated. Machine learning models (e.g., convolutional neural networks) may also segment/locate immune cells which would allow metrics of density and infiltration to be calculated. A wide variety of other tumor microenvironment (TME) features may be generated using a suite of machine learning models, including tumor histology type, tumor cell type, stroma, normal cell types, necrosis, different immune cell types, etc. Some of these features could also be generated using traditional image processing instead of or in addition to machine learning learning approaches.

Many of these features can be quantified to generate tumor microenvironment profiles for localized (or global) tissue regions on a slide. The system can generate a tumor microenvironment profile for each region, which can comprise a collection of metrics, from which the system can compare localized regions to each other. For example, the density of tumor infiltrating lymphocytes (TILs) may be different from one tumor region to another. Measures of tumor microenvironment heterogeneity (such as immune density of infiltration) may be biomarkers for treatment response efficacy and duration. For example, immunotherapy response duration may be shorter or the response may be partial if there is only strong immune infiltration in some areas and not others. A combination of these TME features can be composed into a single score: for example, the system can calculate the spread (variance, interquartile range or IQR, etc.) for each metric and combine these together for a final TME heterogeneity score.

FIG. 5 illustrates an exemplary process for determining a phenotypic tumor heterogeneity score in a sample from an individual, in accordance with some embodiments. Process 500 can be performed, for example, using one or more electronic devices implementing a software platform. In some examples, process 500 is performed using a client-server system, and the blocks of process 500 are divided up in any manner between the server and a client device. In other examples, the blocks of process 500 are divided up between the server and multiple client devices. Thus, while portions of process 500 are described herein as being performed by particular devices of a client-server system, it will be appreciated that process 500 is not so limited. In other examples, process 500 is performed using only a client device or only multiple client devices. In process 500, some blocks are, optionally, combined, the order of some blocks is, optionally, changed, and some blocks are, optionally, omitted. In some examples, additional steps may be performed in combination with the process 500. Accordingly, the operations as illustrated (and described in greater detail below) are exemplary by nature and, as such, should not be viewed as limiting.

At block 502, an exemplary system obtains an image associated with the sample from the individual. In some embodiments, the image is a medical image. In some examples, the image comprises a histopathology image such as an immunohistochemistry (IHC) stain image, a hematoxylin and eosin (H&E) stain image, or any combination thereof.

At block 504, the system inputs the medical image into a trained machine learning model to identify a plurality of phenotypic features in the medical image. The plurality of phenotypic features can comprise: polarity, concavity, texture, shape, and/or size of one or more cells in the sample.

In some embodiments, the trained machine learning model comprises: a segmentation model, a classification model, an object detection model, or any combination thereof. The machine learning model can comprise one or more models that are cell-level focused. For example, to determine the cellular features (size, polarity, concavity, texture, etc.) of the tumor cells on a slide, the system can train an object semantic segmentation model. The object semantic segmentation model can segment out objects-of-interest (e.g., cell types of interest such as tumor cells and lymphocytes) at a pixel level and also predict a class label for each segmented object. The system can then directly calculate the cell features based on the masks of the cells from the segmentation model. For example, the system can mask out tumor cells from the original image and calculate size, shape, texture, etc.

In some embodiments, the trained machine learning model comprises one or more tissue-region focused models that make categorizations for patches or patch groups (which can be obtained using steps 506 and 508. For example, the system can train a model to predict whether a tissue patch is stroma. The system can then generate a prediction map for the whole slide and draw the stroma regions, which can then be used to calculate downstream features such as density (e.g., total stromal area, stroma area bordered by tumor masses, fibroblast density in stroma, etc.) in step 510.

At block 506, the system identifies a plurality of patches from the medical image. In some embodiments, the system may first identify a tumor tissue depicted in the medical image, for example, by inputting the medical image into a trained segmentation machine learning model. The system may then divide the portion of the image depicting the tumor tissue into a plurality of patches. Further exemplary details for identifying the plurality of patches in the medical image are provided herein with reference to FIG. 2A.

At block 508, the system identifies a plurality of patch groups. Each patch group comprises one or more patches of the plurality of patches and corresponds to a region of interest in the medical image. In some embodiments, the plurality of regions of interest may correspond to different visually/morphological distinct portions of a tumor tissue in the medical image. In some embodiments, the plurality of regions of interest may correspond to different local neighborhoods of the tumor tissue in the medical image. In some embodiments, the plurality of regions of interest may correspond to the individual patches. Exemplary methods for identifying the plurality of regions of interest are provided herein with reference to FIG. 2B.

At block 510, the system obtains, based on the plurality of phenotypic features, a plurality of microenvironment profiles corresponding to the plurality of patch groups. In some embodiments, each microenvironment profile of the plurality of microenvironment profiles can comprise: presence, density, and/or infiltration of one or more immune cells, a tumor histology type, a tumor cell type, stroma, a normal cell type, necrosis, an immune cell type (e.g., macrophages, T cells), a group-to-group ratio (e.g., ratio of immune cells to tumor cells in a given area), or any combination thereof. In some embodiments, one or more of the metrics can be obtained using one or more machine learning models.

At block 512, the system generates the phenotypic tumor heterogeneity score by comparing the plurality of microenvironment profiles. In some embodiments, the system calculates a variance value based on the plurality of microenvironment profiles. For example, the system can calculate the spread (variance, IQR, etc.) for each metric of the microenvironment profile and combine these together for a final TME heterogeneity score. For example, the system assigns the phenotypic tumor heterogeneity score to a first value indicative of lower tumor heterogeneity if the variance value is lower than or equal to a predefined threshold and assigns the phenotypic tumor heterogeneity score to a second value indicative of higher tumor heterogeneity if the variance value is higher than a predefined threshold. As another example, the phenotypic tumor heterogeneity score can be proportional to the variance value.

The phenotypic tumor heterogeneity score can be combined with a genomic tumor heterogeneity score to generate a composite tumor heterogeneity score. The genomic tumor heterogeneity score may be obtained from sequencing data associated with the same sample depicted in the image and/or a different sample of the subject. The composite tumor heterogeneity score can be calculated based on the phenotypic tumor heterogeneity score and the genomic tumor heterogeneity score. In some embodiments, the composite tumor heterogeneity score may be a weighted average of the phenotypic tumor heterogeneity score and the genomic tumor heterogeneity score. In some embodiments, the composite tumor heterogeneity score may be a composite binarization or a function of a categorical phenotypic tumor heterogeneity score and a categorical genomic tumor heterogeneity score. Alternatively, the composite tumor heterogeneity score may be a binarization of the phenotypic tumor heterogeneity score and the genomic tumor heterogeneity score. For example, if both the genomic tumor heterogeneity score and the phenotypic tumor heterogeneity score are indicative of high heterogeneity, then the system can assign the composite tumor heterogeneity score to be indicative of high heterogeneity.

The composite tumor heterogeneity score can be used for a number of downstream applications. In some embodiments, the composite tumor heterogeneity score can be used to identify a combination of treatments for the individual. For example, if the composite tumor heterogeneity score is indicative of high heterogeneity, the system may determine a combination of treatment for the individual. As another example, if the composite tumor heterogeneity score is indicative of high heterogeneity, the system may provide a recommendation to monitor the subject and/or the subject's response to treatments earlier and/or more frequently.

In some embodiments, the composite tumor heterogeneity score can be used to predict the duration of a therapeutic response to the anti-cancer therapy. For example, if the composite tumor heterogeneity score is less than or equal to a threshold tumor heterogeneity score, the system may predict the individual to have a longer duration of therapeutic response to an anti-cancer therapy. On the other hand, if the composite tumor heterogeneity score is more than the threshold tumor heterogeneity score, the system may the individual to have a shorter duration of therapeutic response to the anti-cancer therapy.

In some embodiments, the composite tumor heterogeneity score can be used to predict the survival rate of the subject if treated with an anti-cancer therapy. For example, if the composite tumor heterogeneity score is less than or equal to the threshold tumor heterogeneity score, the system may predict the individual to have a higher survival rate if treated with an anti-cancer therapy. On the other hand, if the composite tumor heterogeneity score is more than the threshold tumor heterogeneity score, the system may predict the individual to have a lower survival rate if treated with the anti-cancer therapy.

In some embodiments, the phenotypic tumor heterogeneity score may be determined before the genomic tumor heterogeneity score is determined. This may be because the sequencing data of the subject, which is needed to calculate the genomic tumor heterogeneity score, may take longer time to obtain than histopathology images of the subject. Accordingly, the phenotypic tumor heterogeneity score may be used for early decision-making before samples of the subject are sequenced and/or before the genomic tumor heterogeneity score is available. The phenotypic tumor heterogeneity score may be used for any of the downstream applications described above by itself. For example, the system can determine a monitoring plan for the individual based on the phenotypic tumor heterogeneity score before sequencing the sample from the individual.

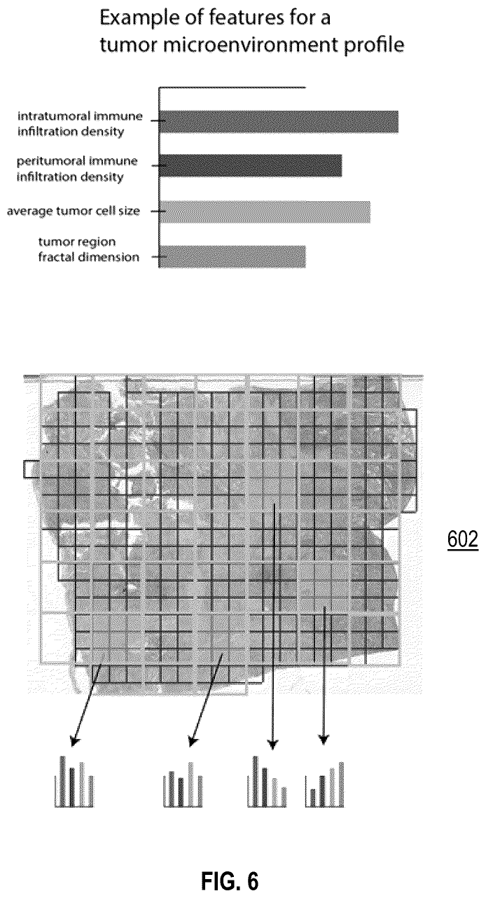

FIG. 6 illustrates an exemplary process for determining phenotypic/morphological tumor heterogeneity based on tumor microenvironment profiles, in accordance with some embodiments. A histopathology whole slide image 602 can be divided into a plurality of patches, as indicated by the black grids. The system can further identify of plurality of patch groups, as indicated by the green grids. In the depicted example, four microenvironment profiles are generated for four patch groups, respectively. Each tumor microenvironment profile comprises a plurality of features as shown in FIG. 6. The tumor microenvironment profiles of different patch groups can be compared to determine phenotypic tumor heterogeneity.

### Methods for Determining Tumor Heterogeneity Based on Embeddings

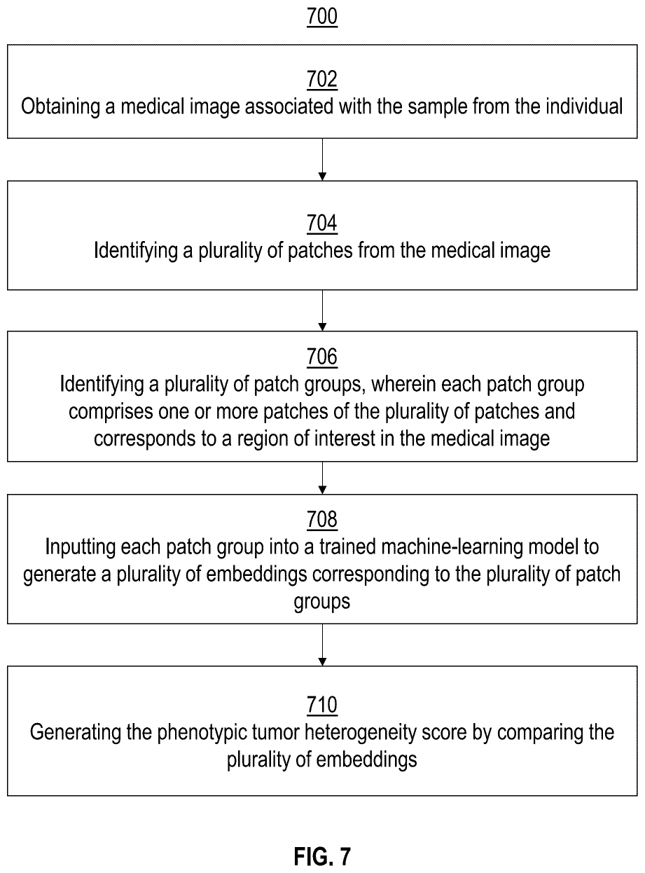

FIG. 7 illustrates an exemplary process for determining a phenotypic tumor heterogeneity score in a sample from an individual, in accordance with some embodiments. Process 700 can be performed, for example, using one or more electronic devices implementing a software platform. In some examples, process 700 is performed using a client-server system, and the blocks of process 700 are divided up in any manner between the server and a client device. In other examples, the blocks of process 700 are divided up between the server and multiple client devices. Thus, while portions of process 700 are described herein as being performed by particular devices of a client-server system, it will be appreciated that process 700 is not so limited. In other examples, process 700 is performed using only a client device or only multiple client devices. In process 700, some blocks are, optionally, combined, the order of some blocks is, optionally, changed, and some blocks are, optionally, omitted. In some examples, additional steps may be performed in combination with the process 700. Accordingly, the operations as illustrated (and described in greater detail below) are exemplary by nature and, as such, should not be viewed as limiting.

At block 702, an exemplary system (e.g., one or more electronic devices) obtains an image associated with the sample from the individual. In some embodiments, the image is a medical image. In some examples, the image comprises a histopathology image such as an immunohistochemistry (IHC) stain image, a hematoxylin and eosin (H&E) stain image, or any combination thereof.

At block 704, the system identifies a plurality of patches from the medical image. In some embodiments, the system may first identify a tumor tissue depicted in the medical image, for example, by inputting the medical image into a trained segmentation machine learning model. The system may then divide the portion of the image depicting the tumor tissue into a plurality of patches. Further exemplary details for identifying the plurality of patches in the medical image are provided herein with reference to FIG. 2A.

At block 706, the system identifies a plurality of patch groups. Each patch group comprises one or more patches of the plurality of patches and corresponds to a region of interest in the medical image. In some embodiments, the plurality of regions of interest may correspond to different visually distinct portions of a tumor tissue in the medical image. In some embodiments, the plurality of regions of interest may correspond to different local neighborhoods of the tumor tissue in the medical image. In some embodiments, the plurality of regions of interest may correspond to the individual patches. Exemplary methods for identifying the plurality of regions of interest are provided herein with reference to FIG. 2B.

At block 708, the system inputs each patch group into a trained machine learning model to generate a plurality of embeddings corresponding to the plurality of patch groups. The machine learning model can be configured to receive a patch group (e.g., in its entirety, patch by patch) and output an embedding corresponding to the patch group, which is a vector representation of the patch group. In some embodiments, the system can obtain patch-level embeddings for all patches in a patch group, and then aggregate the patch-level embeddings to obtain a patch-group-level embedding.

In some embodiments, the trained machine learning model comprises an unsupervised model or self-supervised model. In some embodiments, the trained machine learning model comprises an autoencoder (e.g., the encoder component), a computer vision model, a discriminator component of a generative adversarial network model (GAN), or any combination thereof.

In some embodiments, the trained machine learning model may comprise a pretrained model. For example, the model may be pretrained on one or more upstream tasks (e.g., pretext tasks). Pretext tasks can allow the model to learn useful feature representations or model weights. For example, the pretrained model may be a convolutional neural network or a visual transformer that has learned core visual concepts through one or more upstream tasks. In some embodiments, the pretext tasks comprise image classification, object detection, image segmentation, etc. In some embodiments, the upstream tasks can be unsupervised in nature, and the goal of the model is to understand semantic meaning of individual visual concepts and encode them to a lower-dimensionality feature space. In some embodiments, the model can comprise autoencoders, which explicitly minimize reconstruction error of an encoder-decoder architecture wherein the encoder is configured to transform an input image into a vector in a latent space and the decoder is configured to transform a vector in the latent space into an image (e.g., back to the input image). In some embodiments, the model can comprise standard computer vision models (e.g., ResNet) as backbones and the weights are trained to understand the visual space of the relevant domain by performing self-supervision tasks (e.g., matching visually similar images, predicting rotations, etc.). These models can be trained on visual domains separate from histopathology and/or directly on histopathology images.

In some embodiments, the embedding can provide a highly compressed representation of the raw image data. Through training, the model can develop a semantic understanding of the domain it has been trained on. For example, input images that are semantically/visually similar would map into the latent space (i.e., the feature embedding space) to embedding points that are closer together than embedding points from more dissimilar input images. This allows for a quantitative assessment of how similar or dissimilar (according to the feature embedding space) different intra-tumoral tissue regions are in block 710.

At block 710, the system generates the phenotypic tumor heterogeneity score by comparing the plurality of embeddings. In some embodiments, the system can compare two embeddings by calculating a distance measure (e.g., the Euclidean distance) between the embeddings. A higher distance value between two patch groups can be indicative of a bigger difference between the corresponding regions of interest (i.e., higher heterogeneity). It should be appreciated that other metrics can be used to quantify the difference between two embeddings. In some embodiments, the system can generate a variance value based on multiple distance values (e.g., by averaging the distance values). In some embodiments, the system can generate the phenotypic tumor heterogeneity score by selecting one or more feature values from each embedding of the plurality of embeddings for comparison. For example, the system can analyze specific features within the embeddings, identify what semantic concepts they capture, and choose to compare only the biologically relevant features when calculating the heterogeneity.

The phenotypic tumor heterogeneity score can be combined with a genomic tumor heterogeneity score to generate a composite tumor heterogeneity score. The genomic tumor heterogeneity score may be obtained from sequencing data associated with the same sample depicted in the image and/or a different sample of the subject. The composite tumor heterogeneity score can be calculated based on the phenotypic tumor heterogeneity score and the genomic tumor heterogeneity score. In some embodiments, the composite tumor heterogeneity score may be a weighted average of the phenotypic tumor heterogeneity score and the genomic tumor heterogeneity score. In some embodiments, the composite tumor heterogeneity score may be a composite binarization or a function of a categorical phenotypic tumor heterogeneity score and a categorical genomic tumor heterogeneity score. Alternatively, the composite tumor heterogeneity score may be a binarization of the phenotypic tumor heterogeneity score and the genomic tumor heterogeneity score. For example, if both the genomic tumor heterogeneity score and the phenotypic tumor heterogeneity score are indicative of high heterogeneity, then the system can assign the composite tumor heterogeneity score to be indicative of high heterogeneity.

The composite tumor heterogeneity score can be used for a number of downstream applications. In some embodiments, the composite tumor heterogeneity score can be used to identify a combination of treatments for the individual. For example, if the composite tumor heterogeneity score is indicative of high heterogeneity, the system may determine a combination of treatment for the individual. As another example, if the composite tumor heterogeneity score is indicative of high heterogeneity, the system may provide a recommendation to monitor the subject and/or the subject's response to treatments earlier and/or more frequently.

In some embodiments, the composite tumor heterogeneity score can be used to predict the duration of a therapeutic response to the anti-cancer therapy. For example, if the composite tumor heterogeneity score is less than or equal to a threshold tumor heterogeneity score, the system may predict the individual to have a longer duration of therapeutic response to an anti-cancer therapy. On the other hand, if the composite tumor heterogeneity score is more than the threshold tumor heterogeneity score, the system may the individual to have a shorter duration of therapeutic response to the anti-cancer therapy.

In some embodiments, the composite tumor heterogeneity score can be used to predict the survival rate of the subject if treated with an anti-cancer therapy. For example, if the composite tumor heterogeneity score is less than or equal to the threshold tumor heterogeneity score, the system may predict the individual to have a higher survival rate if treated with an anti-cancer therapy. On the other hand, if the composite tumor heterogeneity score is more than the threshold tumor heterogeneity score, the system may predict the individual to have a lower survival rate if treated with the anti-cancer therapy.

In some embodiments, the phenotypic tumor heterogeneity score may be determined before the genomic tumor heterogeneity score is determined. This may be because the sequencing data of the subject, which is needed to calculate the genomic tumor heterogeneity score, may take longer time to obtain than histopathology images of the subject. Accordingly, the phenotypic tumor heterogeneity score may be used for early decision-making before samples of the subject are sequenced and/or before the genomic tumor heterogeneity score is available. The phenotypic tumor heterogeneity score may be used for any of the downstream applications described above by itself. For example, the system can determine a monitoring plan for the individual based on the phenotypic tumor heterogeneity score before sequencing the sample from the individual.

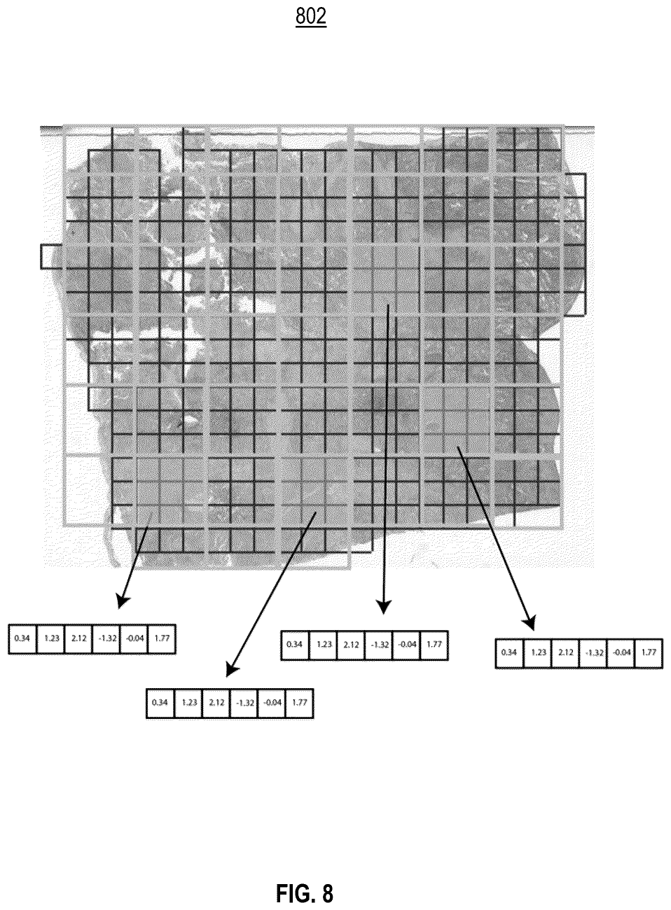

FIG. 8 illustrates an exemplary process for determining phenotypic/morphological tumor heterogeneity based on embeddings, in accordance with some embodiments. A histopathology whole slide image 802 can be divided into a plurality of patches, as indicated by the black grids. The system can further identify of plurality of patch groups, as indicated by the green grids. In the depicted example, four embeddings are generated for four patch groups, respectively. Each embedding comprises a vector of features as shown in FIG. 8. The embeddings of different patch groups can be compared to determine phenotypic tumor heterogeneity.

In some instances, the gene panel may comprise at least 1, at least 2, at least 3, at least 4, at least 5, at least 6, at least 7, at least 8, at least 9, at least 10, at least 20, at least 30, at least 40, or more than 40 genes.

In some instances, the disclosed methods may be used to identify variants in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLDI, POLE, PPARG, PPP2RIA, PPP2R2A, PRDM1, PRKAR1A, PRKC1, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof.

In some instances, the disclosed methods may be used to identify variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene locus, or any combination thereof.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, (viii) combining the nucleic acid sequence data (including, e.g., variant data, copy number data, methylation status data, etc., of the sequenced nucleic acid molecules) with other biomarker data modalities including, but not limited to, proteomics-based biomarker data (e.g., the detection of specific polypeptides, such as proteins) or fragmentomics-based biomarker data (e.g., the detection of certain attributes related to nucleic acid fragments, such as fragment size or the sequences of fragment ends), to determine, for example, the presence of ctDNA in the sample and/or to determine a diagnostic, prognostic, and/or treatment response prediction for the subject, and (ix) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA). In some instances, the cell-free DNA (cfDNA), or a portion thereof, may comprise circulating tumor DNA (ctDNA). In some instances, the sample may be a liquid biopsy sample and may comprise a combination of cell-free DNA (cfDNA) and circulating tumor DNA (ctDNA).

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for determining tumor heterogeneity (e.g., phenotypic score, genomic score, composite score) may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for determining tumor heterogeneity (e.g., phenotypic score, genomic score, composite score) may be used to predict genetic disorders in fetal DNA. (e.g., for invasive or non-invasive prenatal testing). For example, sequence read data obtained by sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify variants, e.g., copy number alterations, associated with, e.g., Down Syndrome (trisomy 21), trisomy 18, trisomy 13, and extra or missing copies of the X and Y chromosomes.

In some instances, the disclosed methods for determining tumor heterogeneity (e.g., phenotypic score, genomic score, composite score) may be used to select a subject (e.g., a patient) for a clinical trial. In some instances, patient selection for clinical trials based on, e.g., identification of tumor heterogeneity, may accelerate the development of targeted therapies and improve the healthcare outcomes for treatment decisions.

In some instances, the disclosed methods for determining tumor heterogeneity (e.g., phenotypic score, genomic score, composite score) may be used to select an appropriate therapy or treatment (e.g., an anti-cancer therapy or anti-cancer treatment) for a subject. In some instances, for example, the anti-cancer therapy or treatment may comprise use of a poly(ADP-ribose) polymerase inhibitor (PARPi), a platinum compound, chemotherapy, radiation therapy, a targeted therapy, an immunotherapy, a neoantigen-based therapy, surgery, or any combination thereof.

In some instances, the anti-cancer therapy or treatment may comprise a targeted anti-cancer therapy or treatment (e.g., a monoclonal antibody-based therapy, an enzyme inhibitor-based therapy, an antibody-drug conjugate therapy, a hormone therapy, and/or a targeted radiotherapy) that targets specific molecules required for cancer cell growth, division, and spreading. In some instances, the targeted anti-cancer therapy or treatment may comprise abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the anti-cancer therapy or treatment may comprise an immunotherapy (e.g., a cancer treatment that acts by stimulating the immune system to fight cancer). In some instances, the immunotherapy can be, for example, an immune system modulator (e.g., a cytokine, such as an interferon or interleukin), an immune checkpoint inhibitor (such as an anti-PD-1 or anti-PD-L1 antibody), a T-cell transfer therapy (e.g., a tumor infiltrating lymphocyte (TIL) therapy in lymphocytes extracted from a patient's tumor are selected for their ability to recognize tumor cells and propagated prior to reintroduction into the patient, or a CAR T-cell therapy in which a patient's T-cells are modified to express the CAR protein prior to reintroduction into the patient), a monoclonal antibody-based therapy (e.g., a monoclonal antibody that binds to cell surface markers on cancer cells to facilitate recognition by the immune system), or a cancer treatment vaccine (e.g., a vaccine based on tumor cells, tumor-associated neoantigens, or dendritic cells, etc., that stimulates the immune system to fight cancer).

In some instances, the anti-cancer therapy or treatment may comprise a neoantigen-based therapy. Non-limiting examples of neoantigen-based therapies include T-cell receptor (TCR) engineered T-cell (TCR-T) therapies, chimeric antigen receptor T-cell (CAR-T) therapies, TCR bispecific antibody therapies, and cancer vaccines. TCR-T therapies are produced by genetically engineering a patient's T-cells to express T-cell receptors that are specific to neoantigens of interest, and then infusing them back into the patient. CAR-T therapies are produced by genetically engineering a patient's T-cells to express chimeric antigen receptor molecules which contain an intracellular signaling and co-signaling domain as well as an extracellular antigen-binding domain; CAR-T therapies don't always rely on neoantigen presentation, but can be designed to be directed towards neoantigens. TCR bispecific antibody therapies are small, engineered antibody molecules that comprise a neoantigen-specific TCR on one end and a CD3-directed single-chain variable fragment on the other end. Cancer vaccines can include RNA molecules, DNA molecules, peptides, or a combination thereof that are designed to boost the immune system's ability to find and destroy neoantigen-presenting cells.

In some instances, the disclosed methods for determining tumor heterogeneity (e.g., phenotypic score, genomic score, composite score) may be used in treating a disease (e.g., a cancer) in a subject. For example, in response to determining tumor heterogeneity (e.g., phenotypic score, genomic score, composite score) using any of the methods disclosed herein, an effective amount of an anti-cancer therapy or anti-cancer treatment may be administered to the subject.

In some instances, the disclosed methods for determining tumor heterogeneity (e.g., phenotypic score, genomic score, composite score) may be used for monitoring disease progression or recurrence (e.g., cancer or tumor progression or recurrence) in a subject. For example, in some instances, the methods may be used to determine determining tumor heterogeneity (e.g., phenotypic score, genomic score, composite score) in a first sample obtained from the subject at a first time point, and used to determine determining tumor heterogeneity (e.g., phenotypic score, genomic score, composite score) in a second sample obtained from the subject at a second time point, where comparison of the first determination of tumor heterogeneity and the second determination of tumor heterogeneity allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of tumor heterogeneity.

In some instances, the value of tumor heterogeneity determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for determining tumor heterogeneity (e.g., phenotypic score, genomic score, composite score) may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for determining tumor heterogeneity as part of a genomic profiling process (or inclusion of the output from the disclosed methods for determining tumor heterogeneity as part of the genomic profile of the subject) can improve the validity of, e.g., disease detection calls and treatment decisions, made on the basis of the genomic profile by, for example, independently confirming the presence of tumor heterogeneity in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods.

In other instances, the cDNA synthesis is initiated at the poly(A) tail of mature mRNAs by priming with oligo (dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164 (1): 35-42; Masuda, et al., (1999) Nucleic Acids Res. 27 (22): 4436-4443; Specht, et al., (2001) Am J Pathol. 158 (2): 419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4 (11): 903-5; Hodges, E. et al. (2007) Nat. Genet. 39 (12): 1522-7; and Okou, D. T. et al. (2007) Nat. Methods 4 (11): 907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 103, 104, 105 or more than 105 molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147 (1): 195-197), the Striped Smith-Waterman algorithm (see, e.g., Farrar (2007), “Striped Smith-Waterman Speeds Database Searches Six Times Over Other SIMD Implementations”, Bioinformatics 23 (2): 156-161), the Needleman-Wunsch algorithm (Needleman, et al. (1970) “A General Method Applicable to the Search for Similarities in the Amino Acid Sequence of Two Proteins”, J. Molecular Biology 48 (3): 443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. CàT in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Alignment of Methyl-Seq Sequence Reads

In some instances, the methods may include the use of an alignment method optimized for aligning sequence reads for DNA that has been converted using, e.g., a bisulfite reaction, to convert unmethylated cytosine residues to uracil (which is interpreted as a thymine in sequencing results). In some instances, sequence reads may be aligned to two genomes in silico, e.g., converted and unconverted versions of the reference genome, using such alignment tools. Methylation occurs primarily at CpG sites, but may also occur less frequently at non-CpG sites (e.g., CHG or CHH sites).

In some instances, the sequence read data may be obtained using a nucleic acid sequencing method comprising the use of a bisulfite- or enzymatic-conversion reaction (e.g., during library preparation) to convert non-methylated cytosine to uracil (see, e.g., Li, et al. (2011), “DNA Methylation Detection: Bisulfite Genomic Sequencing Analysis”, Methods Mol. Biol. 791:11-21).

In some instances, the sequence read data may be obtained using a nucleic acid sequencing method comprising the use of alternative chemical and/or enzymatic reactions (e.g., during library preparation) to convert non-methylated cytosine to uracil (or to convert methylated cytosine to dihydrouracil). For example, enzymatic deamination of non-methylated cytosine using APOBEC to form uracil can be performed using, e.g., the Enzymatic Methyl-seq Kit from New England BioLabs (Ipswich, MA) which uses prior treatment with ten-eleven translocation methylcytosine dioxygenase 2 (TET2) to oxidize 5-mC and 5-hmC, thereby providing greater protection of the methylated cytosine from deamination by APOBEC). Liu, et al. (2019) recently described a bisulfite-free and base-level-resolution sequencing-based method, TET-Assisted Pyridine borane Sequencing (TAPS), for detection of 5mC and 5hmC. The method combines ten-eleven translocation methylcytosine dioxygenase (TET)-mediated oxidation of 5mC and 5hmC to 5-carboxylcytosine (5caC) with pyridine borane reduction of 5caC to dihydrouracil (DHU). Subsequent PCR amplification converts DHU to thymine, thereby enabling conversion of methylated cytosines to thymine (Liu, et al. (2019), “Bisulfite-Free Direct Detection of 5-Methylcytosine and 5-Hydroxymethylcytosine at Base Resolution”, Nature Biotechnology, vol. 37, pp. 424-429).

In some instances, the sequence read data may be obtained using a nucleic acid sequencing method comprising the use of Methylated DNA Immunoprecipitation (MeDIP).

Examples of alignment tools optimized for aligning sequence reads for converted DNA include, but are not limited to, NovoAlign (Novocraft Technologies, Selangor, Malaysia), and the Bismark tool (Krueger, et al. (2011), “Bismark: A Flexible Aligner and Methylation Caller for Bisulfite-Seq Applications”, Bioinformatics 27 (11): 1571-1572).

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26 (5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85 (6): 847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20 (9): 1297-303; Ye, K., et al., Bioinformatics, 2009; 25 (21): 2865-71; Lunter, G., and Goodson, M., Genome Res. 2011; 21 (6): 936-9; and Li, H., et al. (2009), Bioinformatics 25 (16): 2078-9.

Methods for generating indel calls and individual-level genotype likelihoods include, e.g., the Dindel algorithm (Albers, C. A., et al., Genome Res. 2011; 21 (6): 961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21 (6): 952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26 (6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Methylation Status Calling

In some instances, the methods described herein may comprise the use of a methylation status calling method, e.g., to call the methylation status of the CpG sites based on the sequence reads and fragments (complementary pairs of forward and reverse sequence reads) derived from DNA that has been subjected to a chemical or enzymatic conversion reaction, e.g., to convert unmethylated cytosine residues to uracil (which is interpreted as a thymine in sequencing results). Examples of such methylation status calling tools include, but are not limited to, the Bismark tool (Krueger, et al. (2011), “Bismark: A Flexible Aligner and Methylation Caller for Bisulfite-Seq Applications”, Bioinformatics 27 (11): 1571-1572), TARGOMICS (Garinet, et al. (2017), “Calling Chromosome Alterations, DNA Methylation Statuses, and Mutations in Tumors by Simple Targeted Next-Generation Sequencing-A Solution for Transferring Integrated Pangenomic Studies into Routine Practice?”, J. Molecular Diagnostics 19 (5): 776-787), Bicycle (Grana, et al. (2018) “Bicycle: A Bioinformatics Pipeline to Analyze Bisulfite Sequencing Data”, Bioinformatics 34 (8): 1414-5), SMAP (Gao, et al. (2015), “SMAP: A Streamlined Methylation Analysis Pipeline for Bisulfite Sequencing”, Gigascience 4:29), and MeDUSA (Wilson, et al. (2016), “Computational Analysis and Integration of MeDIP-Seq Methylome Data”, in: Kulski J K, editor, Next Generation Sequencing: Advances, Applications and Challenges. Rijeka: InTech, p. 153-69). See also, Rauluseviciute, et al. (2019), “DNA Methylation Data by Sequencing: Experimental Approaches and Recommendations for Tools and Pipelines for Data Analysis”, Clinical Epigenetics 11:193.

### Systems

Also disclosed herein are systems designed to implement any of the disclosed methods for determining a phenotypic tumor heterogeneity score in a sample from a subject. The systems may comprise, e.g., one or more processors, and a memory unit communicatively coupled to the one or more processors and configured to store instructions that, when executed by the one or more processors, cause the system to: obtain a medical image associated with the sample from the individual; identify a plurality of patches from the medical image; identify a plurality of patch groups, wherein each patch group comprises one or more patches of the plurality of patches and corresponds to a region of interest in the medical image; input each patch group into a trained machine learning model to generate a plurality of genomic alteration predictions corresponding to the plurality of patch groups, wherein the plurality of genomic alteration predictions is related to the presence of one or more genomic alterations in each of the input patch groups; and generate the phenotypic tumor heterogeneity score by comparing the plurality of genomic alteration predictions.

Also disclosed herein are systems designed to implement any of the disclosed methods for determining a phenotypic tumor heterogeneity score in a sample from a subject. The systems may comprise, e.g., one or more processors, and a memory unit communicatively coupled to the one or more processors and configured to store instructions that, when executed by the one or more processors, cause the system to: obtain a medical image associated with the sample from the individual; input the medical image into a trained machine learning model to identify a plurality of phenotypic features in the medical image; identify a plurality of patches from the medical image; identify a plurality of patch groups, wherein each patch group comprises one or more patches of the plurality of patches and corresponds to a region of interest in the medical image; obtain, based on the plurality of phenotypic features, a plurality of microenvironment profiles corresponding to the plurality of patch groups; generate the phenotypic tumor heterogeneity score by comparing the plurality of microenvironment profiles

Also disclosed herein are systems designed to implement any of the disclosed methods for determining a phenotypic tumor heterogeneity score in a sample from a subject. The systems may comprise, e.g., one or more processors, and a memory unit communicatively coupled to the one or more processors and configured to store instructions that, when executed by the one or more processors, cause the system to: obtain a medical image associated with the sample from the individual; identify a plurality of patches from the medical image; identify a plurality of patch groups, wherein each patch group comprises one or more patches of the plurality of patches and corresponds to a region of interest in the medical image; input each patch group into a trained machine learning model to generate a plurality of embeddings corresponding to the plurality of patch groups; generate the phenotypic tumor heterogeneity score by comparing the plurality of embeddings.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for determining tumor heterogeneity in any of a variety of samples as described herein (e.g., a tissue sample, biopsy sample, hematological sample, or liquid biopsy sample derived from the subject).

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of tumor heterogeneity is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Machine Learning

Any of a variety of machine learning approaches & algorithms (where a machine learning model, as referred to herein, comprises a trained machine learning algorithm) may be used in implementing the disclosed methods. For example, the machine learning model may comprise a supervised learning model (i.e., a model trained using labeled sets of training data), an unsupervised learning model (i.e., a model trained using unlabeled sets of training data), a semi-supervised learning model (i.e., a model trained using a combination of labeled and unlabeled training data), a self-supervised learning model, or any combination thereof. In some examples, the machine learning model can comprise a deep learning model (i.e., a model comprising many layers of coupled “nodes” that may be trained in a supervised, unsupervised, or semi-supervised manner).

In some instances, one or more machine learning models (e.g., 1, 2, 3, 4, 5, 6, 7, 8, 9, 10, or more than 10 machine learning models), or a combination thereof, may be utilized to implement the disclosed methods.

In some instances, the one or more machine learning models may comprise statistical methods for analyzing data. The machine learning models may be used for classification and/or regression of data. The machine learning models can include, for example, neural networks, support vector machines, decision trees, ensemble learning (e.g., bagging-based learning, such as random forest, and/or boosting-based learning), k-nearest neighbors algorithms, linear regression-based models, and/or logistic regression-based models. The machine learning models can comprise regularization, such as L1 regularization and/or L2 regularization. The machine learning models can include the use of dimensionality reduction techniques (e.g., principal component analysis, matrix factorization techniques, and/or autoencoders) and/or clustering techniques (e.g., hierarchical clustering, k-means clustering, distribution-based clustering, such as Gaussian mixture models, or density-based clustering, such as DBSCAN or OPTICS). The one or more machine learning models can comprise solving, e.g., optimizing, an objective function over multiple iterations based on a training data set. The iterative solving approach can be used even when the machine learning model comprises a model for which there exists a closed-form solution (e.g., linear regression).

In some instances, the machine learning models can comprise artificial neural networks (ANNs), e.g., deep learning models. For example, the one or more machine learning models/algorithms used for implementing the disclosed methods may include an ANN which can comprise any of a variety of computational motifs/architectures known to those of skill in the art, including, but not limited to, feedforward connections (e.g., skip connections), recurrent connections, fully connected layers, convolutional layers, and/or pooling functions (e.g., attention, including self-attention). The artificial neural networks can comprise differentiable non-linear functions trained by backpropagation.

Artificial neural networks, e.g., deep learning models, generally comprise an interconnected group of nodes organized into multiple layers of nodes. For example, the ANN architecture may comprise at least an input layer, one or more hidden layers (i.e., intermediate layers), and an output layer. The ANN or deep learning model may comprise any total number of layers (e.g., 3, 4, 5, 6, 7, 8, 9, 10, 15, 20, or more than 20 layers in total), and any number of hidden layers (e.g., 1, 2, 3, 4, 5, 6, 7, 8, 9, 10, 15, 20, or more than 20 hidden layers), where the hidden layers function as trainable feature extractors that allow mapping of a set of input data to a preferred output value or set of output values. Each layer of the neural network comprises a plurality of nodes (e.g., at least 10, 25, 50, 75 100, 200, 300, 400, 500, 600, 700, 800, 900, 1000, 2000, 3000, 4000, 5000, 6000, 7000, 8000, 9000, 10,000, or more than 10,000 nodes). A node receives input data (e.g., genomic feature data (such as variant sequence data, methylation status data, etc.), non-genomic feature data (e.g., digital pathology image feature data), or other types of input data (e.g., patient-specific clinical data)) that comes either directly from one or more input data nodes or from the output of one or more nodes in previous layers, and performs a specific operation, e.g., a summation operation. In some cases, a connection from an input to a node is associated with a weight (or weighting factor). In some cases, the node may, for example, sum up the products of all pairs of inputs, Xᵢ, and their associated weights, Wᵢ. In some cases, the weighted sum is offset with a bias, b. In some cases, the output of a node may be gated using a threshold or activation function, ƒ, where ƒ may be a linear or non-linear function. The activation function may be, for example, a rectified linear unit (ReLU) activation function or other function such as a saturating hyperbolic tangent, identity, binary step, logistic, arcTan, softsign, parameteric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network (or other machine learning architecture), can be “taught” or “learned” in a training phase using one or more sets of training data (e.g., 1, 2, 3, 4, 5, or more than 5 sets of training data) and a specified training approach configured to solve, e.g., minimize, a loss function. For example, the adjustable parameters for an ANN (e.g., deep learning model) may be determined based on input data from a training data set using an iterative solver (such as a gradient-based method, e.g., backpropagation), so that the output value(s) that the ANN computes (e.g., a classification of a sample or a prediction of a disease outcome) are consistent with the examples included in the training data set. The training of the model (i.e., determination of the adjustable parameters of the model using an iterative solver) may or may not be performed using the same hardware as that used for deployment of the trained model.

In some instances, the disclosed methods may comprise retraining any of the machine learning models (e.g., iteratively retraining a previously trained model using one or more training data sets that differ from those used to train the model initially). In some instances, retraining the machine learning model may comprise using a continuous, e.g., online, machine learning model, i.e., where the model is periodically or continuously updated or retrained based on new training data. The new training data may be provided by, e.g., a single deployed local operational system, a plurality of deployed local operational systems, or a plurality of deployed, geographically-distributed operational systems. In some instances, the disclosed methods may employ, for example, pre-trained ANNs, and the pre-trained ANNs can be fine-tuned according to an additional dataset that is inputted into the pre-trained ANN.

### Computer Systems and Networks

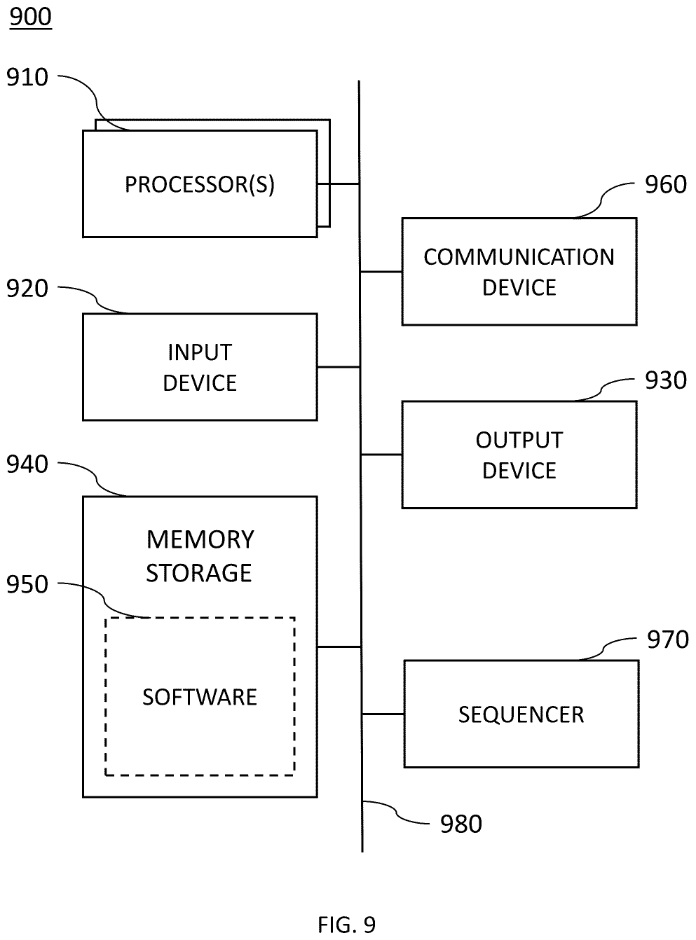

FIG. 9 illustrates an example of a computing device or system in accordance with one embodiment. Device 900 can be a host computer connected to a network. Device 900 can be a client computer or a server. As shown in FIG. 9, device 900 can be any suitable type of microprocessor-based device, such as a personal computer, workstation, server or handheld computing device (portable electronic device) such as a phone or tablet. The device can include, for example, one or more processor(s) 910, input devices 920, output devices 930, memory or storage devices 940, communication devices 960, and nucleic acid sequencers 970. Software 950 residing in memory or storage device 940 may comprise, e.g., an operating system as well as software for executing the methods described herein. Input device 920 and output device 930 can generally correspond to those described herein, and can either be connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage (e.g., an electrical, magnetic or optical memory including a RAM (volatile and non-volatile), cache, hard drive, or removable storage disk). Communication device 960 can include any suitable device capable of transmitting and receiving signals over a network, such as a network interface chip or device. The components of the computer can be connected in any suitable manner, such as via a wired media (e.g., a physical system bus 980, Ethernet connection, or any other wire transfer technology) or wirelessly (e.g., Bluetooth®, Wi-Fi®, or any other wireless technology).

Software module 950, which can be stored as executable instructions in storage 940 and executed by processor(s) 910, can include, for example, an operating system and/or the processes that embody the functionality of the methods of the present disclosure (e.g., as embodied in the devices as described herein).

Software module 950 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 940, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

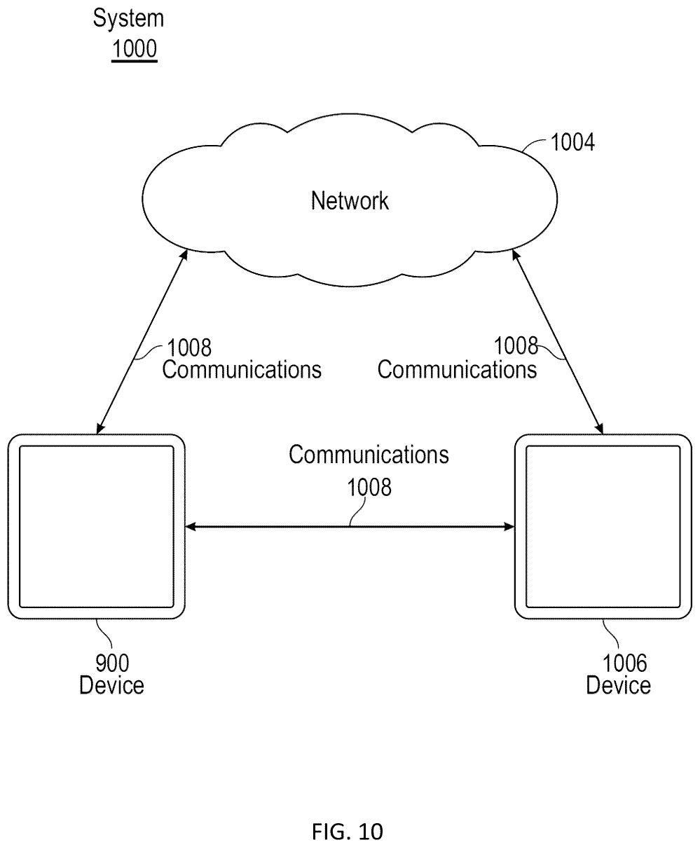

Device 900 may be connected to a network (e.g., network 1004, as shown in FIG. 10 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 900 can be implemented using any operating system, e.g., an operating system suitable for operating on the network. Software module 950 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 910.

Device 900 can further include a sequencer 970, which can be any suitable nucleic acid sequencing instrument.

FIG. 10 illustrates an example of a computing system in accordance with one embodiment. In system 1000, device 900 (e.g., as described above and illustrated in FIG. 9) is connected to network 1004, which is also connected to device 1006. In some embodiments, device 1006 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS system.

Devices 900 and 1006 may communicate, e.g., using suitable communication interfaces via network 1004, such as a Local Area Network (LAN), Virtual Private Network (VPN), or the Internet. In some embodiments, network 1004 can be, for example, the Internet, an intranet, a virtual private network, a cloud network, a wired network, or a wireless network. Devices 900 and 1006 may communicate, in part or in whole, via wireless or hardwired communications, such as Ethernet, IEEE 802.11b wireless, or the like. Additionally, devices 900 and 1006 may communicate, e.g., using suitable communication interfaces, via a second network, such as a mobile/cellular network. Communication between devices 900 and 1006 may further include or communicate with various servers such as a mail server, mobile server, media server, telephone server, and the like. In some embodiments, Devices 900 and 1006 can communicate directly (instead of, or in addition to, communicating via network 1004), e.g., via wireless or hardwired communications, such as Ethernet, IEEE 802.11b wireless, or the like. In some embodiments, devices 900 and 1006 communicate via communications 1008, which can be a direct connection or can occur via a network (e.g., network 1004).

One or all of devices 900 and 1006 generally include logic (e.g., http web server logic) or are programmed to format data, accessed from local or remote databases or other sources of data and content, for providing and/or receiving information via network 1004 according to various examples described herein.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

### Exemplary Implementations

Exemplary implementations of the methods and systems described herein include:

1. A method for determining a composite tumor heterogeneity score in a sample from a subject, comprising:
providing a plurality of nucleic acid molecules obtained from the sample from the subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
receiving, at one or more processors, sequence read data for the plurality of sequence reads;
generating, using the one or more processors, a genomic tumor heterogeneity score based on the sequence read data;
obtaining a medical image associated with the sample;
identifying a plurality of patches from the medical image;
identifying a plurality of patch groups, wherein each patch group comprises one or more patches of the plurality of patches and corresponds to a region of interest in the medical image;
inputting each patch group into a trained machine learning model to generate a plurality of genomic alteration predictions corresponding to the plurality of patch groups, wherein the plurality of genomic alteration predictions is related to the presence of one or more genomic alterations in each of the input patch groups;
generating a phenotypic tumor heterogeneity score by comparing the plurality of genomic alteration predictions; and
calculating the composite tumor heterogeneity score based on the phenotypic tumor heterogeneity score and the genomic tumor heterogeneity score.

2. The method of clause 1, wherein the medical image comprises a histopathology image.

3. The method of clause 1 or clause 2, wherein the composite tumor heterogeneity score is a weighted average or a binarization of the phenotypic tumor heterogeneity score and the genomic tumor heterogeneity score.

4. The method of any one of clauses 1 to 3, further comprising: identifying a combination of treatments for the subject based on the composite tumor heterogeneity score.

5. The method of any one of clauses 1 to 4, further comprising: determining a monitoring plan for the subject based on the phenotypic tumor heterogeneity score.

6. The method of any one of clauses 1 to 5, further comprising:
if the composite tumor heterogeneity score is less than or equal to a threshold tumor heterogeneity score, predicting the subject to have a longer duration of therapeutic response to an anti-cancer therapy; and
if the composite tumor heterogeneity score is more than the threshold tumor heterogeneity score, predicting the subject to have a shorter duration of therapeutic response to the anti-cancer therapy.

7. The method of any one of clauses 1 to 6, further comprising:
if the composite tumor heterogeneity score is less than or equal to a threshold tumor heterogeneity score, predicting the subject to have a higher survival rate if treated with an anti-cancer therapy; and
if the composite tumor heterogeneity score is more than the threshold tumor heterogeneity score, predicting the subject to have a lower survival rate if treated with the anti-cancer therapy.

8. The method of any one of clauses 1 to 7, wherein the subject is suspected of having or is determined to have cancer.

9. The method of clause 8, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

10. The method of clause 8, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

11. The method of any one of clauses 8 to 10, further comprising treating the subject with an anti-cancer therapy.

12. The method of clause 11, wherein the anti-cancer therapy comprises a targeted anti-cancer therapy.

13. The method of clause 12, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

14. The method of any one of clauses 1 to 13, further comprising obtaining the sample from the subject.

15. The method of any one of clauses 1 to 14, wherein the sample comprises a tissue biopsy sample, a liquid biopsy sample, or a normal control.

16. The method of clause 15, wherein the sample is a liquid biopsy sample and comprises blood, plasma, cerebrospinal fluid, sputum, stool, urine, or saliva.

17. The method of clause 15, wherein the sample is a liquid biopsy sample and comprises circulating tumor cells (CTCs).

18. The method of clause 15, wherein the sample is a liquid biopsy sample and comprises cell-free DNA (cfDNA), circulating tumor DNA (ctDNA), or any combination thereof.

19. The method of any one of clauses 1 to 18, wherein the plurality of nucleic acid molecules comprises a mixture of tumor nucleic acid molecules and non-tumor nucleic acid molecules.

20. The method of clause 19, wherein the tumor nucleic acid molecules are derived from a tumor portion of a heterogeneous tissue biopsy sample, and the non-tumor nucleic acid molecules are derived from a normal portion of the heterogeneous tissue biopsy sample.

21. The method of clause 19, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

22. The method of any one of clauses 1 to 21, wherein the one or more adapters comprise amplification primers, flow cell adaptor sequences, substrate adapter sequences, or sample index sequences.

23. The method of any one of clauses 1 to 22, wherein the captured nucleic acid molecules are captured from the amplified nucleic acid molecules by hybridization to one or more bait molecules.

24. The method of clause 23, wherein the one or more bait molecules comprise one or more nucleic acid molecules, each comprising a region that is complementary to a region of a captured nucleic acid molecule.

25. The method of any one of clauses 1 to 24, wherein amplifying nucleic acid molecules comprises performing a polymerase chain reaction (PCR) amplification technique, a non-PCR amplification technique, or an isothermal amplification technique.

26. The method of any one of clauses 1 to 25, wherein the sequencing comprises use of a massively parallel sequencing (MPS) technique, whole genome sequencing (WGS), whole exome sequencing, targeted sequencing, direct sequencing, or Sanger sequencing technique.

27. The method of clause 26, wherein the sequencing comprises massively parallel sequencing, and the massively parallel sequencing technique comprises next generation sequencing (NGS).

28. The method of any one of clauses 1 to 27, wherein the sequencer comprises a next generation sequencer.

29. The method of any one of clauses 1 to 28, wherein one or more of the plurality of sequencing reads overlap one or more gene loci within one or more subgenomic intervals in the sample.

30. The method of clause 29, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

31. The method of clause 29 or clause 30, wherein the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLDI, POLE, PPARG, PPP2RIA, PPP2R2A, PRDM1, PRKAR1A, PRKC1, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

32. The method of clause 29 or clause 30, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

33. The method of any one of clauses 1 to 32, further comprising generating, by the one or more processors, a report indicating the presence or absence of the genomic alteration.

34. The method of clause 33, further comprising transmitting the report to a healthcare provider.

35. The method of clause 34, wherein the report is transmitted via a computer network or a peer-to-peer connection.

36. A method for determining a phenotypic tumor heterogeneity score in a sample from a subject, comprising:
obtaining a medical image associated with the sample from the subject;
identifying a plurality of patches from the medical image;
identifying a plurality of patch groups, wherein each patch group comprises one or more patches of the plurality of patches and corresponds to a region of interest in the medical image;
inputting each patch group into a trained machine learning model to generate a plurality of genomic alteration predictions corresponding to the plurality of patch groups, wherein the plurality of genomic alteration predictions is related to the presence of one or more genomic alterations in each of the input patch groups; and
generating the phenotypic tumor heterogeneity score by comparing the plurality of genomic alteration predictions.

37. The method of clause 36, wherein the medical image comprises a histopathology image.

38. The method of clause 36 or clause 37, further comprising:
obtaining a genomic tumor heterogeneity score based on sequence read data associated with the sample; and
calculating a composite tumor heterogeneity score based on the phenotypic tumor heterogeneity score and the genomic tumor heterogeneity score.

39. The method of clause 38, wherein the composite tumor heterogeneity score is a weighted average or a binarization of the phenotypic tumor heterogeneity score and the genomic tumor heterogeneity score.

40. The method of clause 38, further comprising: identifying a combination of treatments for the subject based on the composite tumor heterogeneity score.

41. The method of clause 38, further comprising: determining a monitoring plan for the subject based on the phenotypic tumor heterogeneity score before sequencing the sample from the subject.

42. The method of clause 38, further comprising:
if the composite tumor heterogeneity score is less than or equal to a threshold tumor heterogeneity score, predicting the subject to have a longer duration of therapeutic response to an anti-cancer therapy; and
if the composite tumor heterogeneity score is more than the threshold tumor heterogeneity score, predicting the subject to have a shorter duration of therapeutic response to the anti-cancer therapy.

43. The method of clause 38, further comprising:
if the composite tumor heterogeneity score is less than or equal to the threshold tumor heterogeneity score, predicting the subject to have a higher survival rate if treated with an anti-cancer therapy; and
if the composite tumor heterogeneity score is more than the threshold tumor heterogeneity score, predicting the subject to have a lower survival rate if treated with the anti-cancer therapy.

44. The method of any one of clauses 36 to 43, further comprising: identifying a plurality of regions of interest in the medical image.

45. The method of clause 44, wherein identifying the plurality of regions of interest comprises:
inputting the medical image into a trained segmentation machine learning model to identify a tumor tissue depicted in the medical image.

46. The method of clause 45, wherein the plurality of regions of interest corresponds to different visually distinct portions of the tumor tissue in the medical image.

47. The method of clause 45, wherein the plurality of regions of interest corresponds to different local neighborhoods of the tumor tissue in the medical image.

48. The method of any one of clauses 36 to 47, wherein the trained machine learning model is configured to receive a given input patch group and output a single prediction of the presence of a single genomic alteration in the given input patch group.

49. The method of clause 48, wherein the single genomic alteration is: an epidermal growth factor receptor (EGFR) driver mutation. a BRAF driver mutation, a MET driver mutation, an ERBB2 (HER2) driver mutation, a PIK3CA driver mutation, a KRAS driver mutation, an NRAS driver mutation, a BRCA1 driver mutation, a BRCA2 driver mutation, an ATM driver mutation, an FGFR2 driver mutation, a driver mutation in a homologous recombination repair (HRR) gene, an NTRK1 driver mutation, an NTRK2 driver mutation, an NTRK3 driver mutation, an RET driver mutation, an ALK mutation, or a ROS1 mutation.

50. The method of clause 48, wherein generating the phenotypic tumor heterogeneity score comprises:
assigning the phenotypic tumor heterogeneity score to a first value indicative of lower tumor heterogeneity if the plurality of genomic alteration predictions all indicates the presence of the single genomic alteration; and
assigning the phenotypic tumor heterogeneity score to a second value indicative of higher tumor heterogeneity if some of the plurality of genomic alteration predictions indicate the presence of the single genomic alteration and some of the plurality of genomic alteration predictions do not indicate the presence of the single genomic alteration.

51. The method of clause 48, wherein generating the phenotypic tumor heterogeneity score comprises:
calculating a variance value based on the plurality of genomic alteration predictions;
assigning the phenotypic tumor heterogeneity score to a first value indicative of lower tumor heterogeneity if the variance value is lower than or equal to a predefined threshold; and
assigning the phenotypic tumor heterogeneity score to a second value indicative of higher tumor heterogeneity if the variance value is higher than a predefined threshold.

52. The method of any one of clauses 36 to 51, wherein the trained machine learning model is configured to receive a given input patch group and output a plurality of predictions of the presence of a plurality of genomic alterations in the given patch group.

53. The method of clause 52, wherein the plurality of genomic alterations comprises: an epidermal growth factor receptor (EGFR) driver mutation. a BRAF driver mutation, a MET driver mutation, an ERBB2 (HER2) driver mutation, a PIK3CA driver mutation, a KRAS driver mutation, an NRAS driver mutation, a BRCA1 driver mutation, a BRCA2 driver mutation, an ATM driver mutation, an FGFR2 driver mutation, a driver mutation in a homologous recombination repair (HRR) gene, an NTRK1 driver mutation, an NTRK2 driver mutation, an NTRK3 driver mutation, an RET driver mutation, an ALK mutation, a ROSI mutation, or any combination thereof.

54. The method of clause 52, wherein the sum of the plurality of predictions is configured to be 1.

55. The method of clause 52, wherein the plurality of predictions is independent of each other.

56. The method of clause 52, wherein generating the phenotypic tumor heterogeneity score comprises:
generating a plurality of phenotypic tumor heterogeneity sub-scores corresponding to the plurality of genomic alterations, and
calculating the phenotypic tumor heterogeneity score based on the plurality of phenotypic tumor heterogeneity sub-scores.

57. The method of any one of clauses 36 to 56, wherein the trained machine learning model comprises: a convolutional neural network model, a vision transformer model, or any combination thereof.

58. The method of clause 57, wherein the convolutional neural network model comprises an attention mechanism.

59. A method for determining a phenotypic tumor heterogeneity score in a sample from a subject, comprising:
obtaining a medical image associated with the sample from the subject;
inputting the medical image into a trained machine learning model to identify a plurality of phenotypic features in the medical image;
identifying a plurality of patches from the medical image;
identifying a plurality of patch groups, wherein each patch group comprises one or more patches of the plurality of patches and corresponds to a region of interest in the medical image;
obtaining, based on the plurality of phenotypic features, a plurality of microenvironment profiles corresponding to the plurality of patch groups; and
generating the phenotypic tumor heterogeneity score by comparing the plurality of microenvironment profiles.

60. The method of clause 59, wherein the medical image comprises a histopathology image.

61. The method of clause 59 or clause 60, further comprising:
obtaining a genomic tumor heterogeneity score based on sequence read data associated with the sample from the subject; and
calculating a composite tumor heterogeneity score based on the phenotypic tumor heterogeneity score and the genomic tumor heterogeneity score.

62. The method of clause 61, wherein the composite tumor heterogeneity score is a weighted average or a binarization of the phenotypic tumor heterogeneity score and the genomic tumor heterogeneity score.

63. The method of clause 61, further comprising: identifying a combination of treatments for the subject based on the composite tumor heterogeneity score.

64. The method of clause 61, further comprising: determining a monitoring plan for the subject based on the phenotypic tumor heterogeneity score before sequencing the sample from the subject.

65. The method of clause 61, further comprising:
if the composite tumor heterogeneity score is less than or equal to a threshold tumor heterogeneity score, predicting the subject to have a longer duration of therapeutic response to an anti-cancer therapy; and
if the composite tumor heterogeneity score is more than the threshold tumor heterogeneity score, predicting the subject to have a shorter duration of therapeutic response to the anti-cancer therapy.

66. The method of clause 61, further comprising:
if the composite tumor heterogeneity score is less than or equal to the threshold tumor heterogeneity score, predicting the subject to have a higher survival rate if treated with an anti-cancer therapy; and
if the composite tumor heterogeneity score is more than the threshold tumor heterogeneity score, predicting the subject to have a lower survival rate if treated with the anti-cancer therapy.

67. The method of any one of clauses 59 to 66, further comprising: identifying a plurality of regions of interest in the medical image.

68. The method of clause 67, wherein identifying the plurality of regions of interest comprises:
inputting the medical image into a trained segmentation machine learning model to identify a tumor sample depicted in the medical image.

69. The method of clause 68, wherein the plurality of regions of interest corresponds to different visually distinct portions of the tumor sample in the medical image.

70. The method of clause 68, wherein the plurality of regions of interest corresponds to different local neighborhoods of the tumor sample in the medical image.

71. The method of any one of clauses 59 to 70, wherein the plurality of phenotypic features comprises: polarity, concavity, texture, shape, and/or size of one or more cells in the sample.

72. The method of any one of clauses 59 to 71, wherein each microenvironment profile of the plurality of microenvironment profiles comprises: density and/or infiltration of one or more immune cells, a tumor histology type, a tumor cell type, stroma, a normal cell type, necrosis, an immune cell type, a group-to-group ratio, or any combination thereof.

73. The method of any one of clauses 59 to 72, wherein the trained machine learning model comprises: a segmentation model, a classification model, an object detection model, or any combination thereof.

74. The method of clause 71, wherein generating the phenotypic tumor heterogeneity score comprises:
calculating a variance value based on the plurality of microenvironment profiles;
assigning the phenotypic tumor heterogeneity score to a first value indicative of lower tumor heterogeneity if the variance value is lower than or equal to a predefined threshold; and
assigning the phenotypic tumor heterogeneity score to a second value indicative of higher tumor heterogeneity if the variance value is higher than a predefined threshold.

75. A method of determining a phenotypic tumor heterogeneity score in a sample from a subject, comprising:
obtaining a medical image associated with the sample from the subject;
identifying a plurality of patches from the medical image;
identifying a plurality of patch groups, wherein each patch group comprises one or more patches of the plurality of patches and corresponds to a region of interest in the medical image;
inputting each patch group into a trained machine learning model to generate a plurality of embeddings corresponding to the plurality of patch groups; and
generating the phenotypic tumor heterogeneity score by comparing the plurality of embeddings.

76. The method of clause 75, wherein the medical image comprises a histopathology image.

77. The method of clause 75 or clause 76, further comprising:
obtaining a genomic tumor heterogeneity score based on sequence read data associated with the sample from the subject; and
calculating a composite tumor heterogeneity score based on the phenotypic tumor heterogeneity score and the genomic tumor heterogeneity score.

78. The method of clause 77, wherein the composite tumor heterogeneity score is a weighted average or a binarization of the phenotypic tumor heterogeneity score and the genomic tumor heterogeneity score.

79. The method of clause 77, further comprising: identifying a combination of treatments for the subject based on the composite tumor heterogeneity score.

80. The method of clause 77, further comprising: determining a monitoring plan for the subject based on the phenotypic tumor heterogeneity score before sequencing the sample from the subject.

81. The method of clause 77, further comprising:
if the composite tumor heterogeneity score is less than or equal to a threshold tumor heterogeneity score, predicting the subject to have a longer duration of therapeutic response to an anti-cancer therapy; and
if the composite tumor heterogeneity score is more than the threshold tumor heterogeneity score, predicting the subject to have a shorter duration of therapeutic response to the anti-cancer therapy.

82. The method of clause 77, further comprising:
if the composite tumor heterogeneity score is less than or equal to the threshold tumor heterogeneity score, predicting the subject to have a higher survival rate if treated with an anti-cancer therapy; and
if the composite tumor heterogeneity score is more than the threshold tumor heterogeneity score, predicting the subject to have a lower survival rate if treated with the anti-cancer therapy.

83. The method of any one of clauses 75 to 82, further comprising: identifying a plurality of regions of interest in the medical image.

84. The method of clause 83, wherein identifying the plurality of regions of interest comprises: inputting the medical image into a trained segmentation machine learning model to identify a tumor sample depicted in the medical image.

85. The method of clause 84, wherein the plurality of regions of interest corresponds to different visually distinct portions of the tumor sample in the medical image.

86. The method of clause 84, wherein the plurality of regions of interest corresponds to different local neighborhoods of the tumor sample in the medical image.

87. The method of any one of clauses 75 to 86, wherein generating the phenotypic tumor heterogeneity score comprises: calculating a plurality of Euclidean distances, each Euclidean distance representing a difference between two embeddings of the plurality of embeddings.

88. The method of clause 87, wherein generating the phenotypic tumor heterogeneity score comprises: calculating a variance value based on the plurality of Euclidean distances.

89. The method of any one of clauses 75 to 88, wherein generating the phenotypic tumor heterogeneity score comprises:
selecting one or more feature values from each embedding of the plurality of embeddings for comparison.

90. The method of any one of clauses 75 to 89, wherein the trained machine learning model comprises an unsupervised model.

91. The method of clause 90, wherein the trained machine learning model comprises an autoencoder, a computer vision model, or any combination thereof.

92. A method for diagnosing a disease, the method comprising:
diagnosing that a subject has the disease based on a determination of a phenotypic tumor heterogeneity score or a composite tumor heterogeneity score for a sample from the subject, wherein the phenotypic tumor heterogeneity score or the composite tumor heterogeneity score is determined according to the method of any one of the preceding clauses.

93. A method of selecting an anti-cancer therapy, the method comprising:
responsive to determining a phenotypic tumor heterogeneity score or a composite tumor heterogeneity score for a sample from a subject, selecting an anti-cancer therapy for the subject, wherein the phenotypic tumor heterogeneity score or the composite tumor heterogeneity score is determined according to the method of any one of the preceding clauses.

94. A method of treating a cancer in a subject, comprising:
responsive to determining a phenotypic tumor heterogeneity score or a composite tumor heterogeneity score for a sample from the subject, administering an effective amount of an anti-cancer therapy to the subject, wherein the phenotypic tumor heterogeneity score or the composite tumor heterogeneity score is determined according to the method of any one of the preceding clauses.

95. A method for monitoring cancer progression or recurrence in a subject, the method comprising:
determining a first phenotypic tumor heterogeneity score or a first composite tumor heterogeneity score in a first sample obtained from the subject at a first time point according to the method of any one of the preceding clauses;
determining a second phenotypic tumor heterogeneity score or a second composite tumor heterogeneity score in a second sample obtained from the subject at a second time point; and
comparing the first phenotypic tumor heterogeneity score or the first composite tumor heterogeneity score to the second phenotypic tumor heterogeneity score or the second composite tumor heterogeneity score, thereby monitoring the cancer progression or recurrence.

96. The method of clause 95, wherein the second phenotypic tumor heterogeneity score or the second composite tumor heterogeneity score for the second sample is determined according to the method of any one of the preceding clauses.

97. The method of clause 95 or clause 96, further comprising selecting an anti-cancer therapy for the subject in response to the cancer progression.

98. The method of clause 95 or clause 96, further comprising administering an anti-cancer therapy to the subject in response to the cancer progression.

99. The method of clause 95 or clause 96, further comprising adjusting an anti-cancer therapy for the subject in response to the cancer progression.

100. The method of any one of clauses 97 to 99, further comprising adjusting a dosage of the anti-cancer therapy or selecting a different anti-cancer therapy in response to the cancer progression.

101. The method of clause 100, further comprising administering the adjusted anti-cancer therapy to the subject.

102. The method of any one of clauses 95 to 101, wherein the first time point is before the subject has been administered an anti-cancer therapy, and wherein the second time point is after the subject has been administered the anti-cancer therapy.

103. The method of any one of clauses 95 to 101, wherein the subject has a cancer, is at risk of having a cancer, is being routine tested for cancer, or is suspected of having a cancer.

104. The method of any one of clauses 95 to 102, wherein the cancer is a solid tumor.

105. The method of any one of clauses 95 to 102, wherein the cancer is a hematological cancer.

106. The method of any one of clauses 97 to 105, wherein the anti-cancer therapy comprises chemotherapy, radiation therapy, immunotherapy, a targeted therapy, or surgery.

107. The method of any one of clauses 95 to 106, further comprising determining, identifying, or applying the value of the first or second phenotypic tumor heterogeneity score or the first or second composite tumor heterogeneity score for the sample as a diagnostic value associated with the sample.

108. The method of any one of clauses 95 to 107, further comprising generating a genomic profile for the subject based on the determination of the first or second phenotypic tumor heterogeneity score or the first or second composite tumor heterogeneity score.

109. The method of clause 108, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.

110. The method of clause 108 or clause 109, wherein the genomic profile for the subject further comprises results from a nucleic acid sequencing-based test.

111. The method of any one of clauses 108 to 110, further comprising selecting an anti-cancer therapy, administering an anti-cancer therapy, or applying an anti-cancer therapy to the subject based on the generated genomic profile.

112. The method of any one of clauses 95 to 111, wherein the determination of the first or second phenotypic tumor heterogeneity score or the first or second composite tumor heterogeneity score for the sample is used in making suggested treatment decisions for the subject.

113. The method of any one of clauses 95 to 112, wherein the determination of the first or second phenotypic tumor heterogeneity score or the first or second composite tumor heterogeneity score for the sample is used in applying or administering a treatment to the subject.

114. A system comprising:
a) one or more processors; and
b) a memory communicatively coupled to the one or more processors and configured to store instructions that, when executed by the one or more processors, cause the system to:
   i) obtain a medical image associated with a sample from a subject;
   ii) identify a plurality of patches from a medical image;
   iii) identify a plurality of patch groups, wherein each patch group comprises one or more patches of the plurality of patches and corresponds to a region of interest in the medical image;
   iv) input each patch group into a trained machine learning model to generate a plurality of genomic alteration predictions corresponding to the plurality of patch groups, wherein the plurality of genomic alteration predictions is related to the presence of one or more genomic alterations in each of the input patch groups; and
   v) generate a phenotypic tumor heterogeneity score by comparing the plurality of genomic alteration predictions.

115. The system of clause 114, wherein the medical image comprises a histopathology image.

116. The system of clause 114 or clause 115, further comprising instructions that, when executed by the one or more processors, cause the system to:
obtain a genomic tumor heterogeneity score based on sequence read data associated with the sample; and
calculate a composite tumor heterogeneity score based on the phenotypic tumor heterogeneity score and the genomic tumor heterogeneity score.

117. The system of clause 116, wherein the composite tumor heterogeneity score is a weighted average or a binarization of the phenotypic tumor heterogeneity score and the genomic tumor heterogeneity score.

118. The system of clause 116, further comprising: identifying a combination of treatments for the subject based on the composite tumor heterogeneity score.

119. The system of clause 116, further comprising: determining a monitoring plan for the subject based on the phenotypic tumor heterogeneity score before sequencing the sample from the subject.

120. The system of clause 116, further comprising:
if the composite tumor heterogeneity score is less than or equal to a threshold tumor heterogeneity score, predicting the subject to have a longer duration of therapeutic response to an anti-cancer therapy; and
if the composite tumor heterogeneity score is more than the threshold tumor heterogeneity score, predicting the subject to have a shorter duration of therapeutic response to the anti-cancer therapy.

121. The system of clause 116, further comprising:
if the composite tumor heterogeneity score is less than or equal to the threshold tumor heterogeneity score, predicting the subject to have a higher survival rate if treated with an anti-cancer therapy; and
if the composite tumor heterogeneity score is more than the threshold tumor heterogeneity score, predicting the subject to have a lower survival rate if treated with the anti-cancer therapy.

122. A system comprising:
a) one or more processors; and
b) a memory communicatively coupled to the one or more processors and configured to store instructions that, when executed by the one or more processors, cause the system to:
   i) obtain a medical image associated with a sample from a subject;
   ii) input the medical image into a trained machine learning model to identify a plurality of phenotypic features in the medical image;
   iii) identify a plurality of patches from the medical image;
   iv) identify a plurality of patch groups, wherein each patch group comprises one or more patches of the plurality of patches and corresponds to a region of interest in the medical image;
   v) obtain, based on the plurality of phenotypic features, a plurality of microenvironment profiles corresponding to the plurality of patch groups; and
   vi) generate a phenotypic tumor heterogeneity score by comparing the plurality of microenvironment profiles.

123. The system of clause 122, wherein the medical image comprises a histopathology image.

124. The system of clause 122 or clause 123, further comprising instructions that, when executed by the one or more processors, cause the system to:
obtain a genomic tumor heterogeneity score based on sequence read data associated with the sample from the subject; and
calculate a composite tumor heterogeneity score based on the phenotypic tumor heterogeneity score and the genomic tumor heterogeneity score.

125. The system of clause 124, wherein the composite tumor heterogeneity score is a weighted average or a binarization of the phenotypic tumor heterogeneity score and the genomic tumor heterogeneity score.

126. The system of clause 124, further comprising: identifying a combination of treatments for the subject based on the composite tumor heterogeneity score.

127. The system of clause 124, further comprising: determining a monitoring plan for the subject based on the phenotypic tumor heterogeneity score before sequencing the sample from the subject.

128. The system of clause 124, further comprising:
if the composite tumor heterogeneity score is less than or equal to a threshold tumor heterogeneity score, predicting the subject to have a longer duration of therapeutic response to an anti-cancer therapy; and
if the composite tumor heterogeneity score is more than the threshold tumor heterogeneity score, predicting the subject to have a shorter duration of therapeutic response to the anti-cancer therapy.

129. The system of clause 124, further comprising:
if the composite tumor heterogeneity score is less than or equal to the threshold tumor heterogeneity score, predicting the subject to have a higher survival rate if treated with an anti-cancer therapy; and
if the composite tumor heterogeneity score is more than the threshold tumor heterogeneity score, predicting the subject to have a lower survival rate if treated with the anti-cancer therapy.

130. A system comprising:
a) one or more processors; and
b) a memory communicatively coupled to the one or more processors and configured to store instructions that, when executed by the one or more processors, cause the system to:
   i) obtain a medical image associated with a sample from a subject;
   ii) identify a plurality of patches from the medical image;
   iii) identify a plurality of patch groups, wherein each patch group comprises one or more patches of the plurality of patches and corresponds to a region of interest in the medical image;
   iv) input each patch group into a trained machine learning model to generate a plurality of embeddings corresponding to the plurality of patch groups; and
   v) generate a phenotypic tumor heterogeneity score by comparing the plurality of embeddings.

131. The system of clause 130, wherein the medical image comprises a histopathology image.

132. The system of clause 130 or clause 131, further comprising instructions that, when executed by the one or more processors, cause the system to:
obtain a genomic tumor heterogeneity score based on sequence read data associated with the sample from the subject; and
calculate a composite tumor heterogeneity score based on the phenotypic tumor heterogeneity score and the genomic tumor heterogeneity score.

133. The system of clause 132, wherein the composite tumor heterogeneity score is a weighted average or a binarization of the phenotypic tumor heterogeneity score and the genomic tumor heterogeneity score.

134. The system of clause 132, further comprising: identifying a combination of treatments for the subject based on the composite tumor heterogeneity score.

135. The system of clause 132, further comprising: determining a monitoring plan for the subject based on the phenotypic tumor heterogeneity score before sequencing the sample from the subject.

136. The system of clause 132, further comprising:
if the composite tumor heterogeneity score is less than or equal to a threshold tumor heterogeneity score, predicting the subject to have a longer duration of therapeutic response to an anti-cancer therapy; and
if the composite tumor heterogeneity score is more than the threshold tumor heterogeneity score, predicting the subject to have a shorter duration of therapeutic response to the anti-cancer therapy.

137. The system of clause 132, further comprising:
if the composite tumor heterogeneity score is less than or equal to the threshold tumor heterogeneity score, predicting the subject to have a higher survival rate if treated with an anti-cancer therapy; and
if the composite tumor heterogeneity score is more than the threshold tumor heterogeneity score, predicting the subject to have a lower survival rate if treated with the anti-cancer therapy.

138. A non-transitory computer-readable storage medium storing one or more programs, the one or more programs comprising instructions, which when executed by one or more processors of a system, cause the system to:
a) obtain a medical image associated with a sample from a subject;
b) identify a plurality of patches from a medical image;
c) identify a plurality of patch groups, wherein each patch group comprises one or more patches of the plurality of patches and corresponds to a region of interest in the medical image;
d) input each patch group into a trained machine learning model to generate a plurality of genomic alteration predictions corresponding to the plurality of patch groups, wherein the plurality of genomic alteration predictions is related to the presence of one or more genomic alterations in each of the input patch groups; and
e) generate a phenotypic tumor heterogeneity score by comparing the plurality of genomic alteration predictions.

139. The non-transitory computer-readable storage medium of clause 138, wherein the medical image comprises a histopathology image.

140. The non-transitory computer-readable storage medium of clause 138 or 139, further comprising instructions that, when executed by one or more processors of a system, cause the system to:
obtain a genomic tumor heterogeneity score based on sequence read data associated with the sample; and
calculate a composite tumor heterogeneity score based on the phenotypic tumor heterogeneity score and the genomic tumor heterogeneity score.

141. The non-transitory computer-readable storage medium of clause 140, wherein the composite tumor heterogeneity score is a weighted average or a binarization of the phenotypic tumor heterogeneity score and the genomic tumor heterogeneity score.

142. The non-transitory computer-readable storage medium of clause 140, further comprising: identifying a combination of treatments for the subject based on the composite tumor heterogeneity score.

143. The non-transitory computer-readable storage medium of clause 140, further comprising: determining a monitoring plan for the subject based on the phenotypic tumor heterogeneity score before sequencing the sample from the subject.

144. The non-transitory computer-readable storage medium of clause 140, further comprising:
if the composite tumor heterogeneity score is less than or equal to a threshold tumor heterogeneity score, predicting the subject to have a longer duration of therapeutic response to an anti-cancer therapy; and
if the composite tumor heterogeneity score is more than the threshold tumor heterogeneity score, predicting the subject to have a shorter duration of therapeutic response to the anti-cancer therapy.

145. The non-transitory computer-readable storage medium of clause 140, further comprising:
if the composite tumor heterogeneity score is less than or equal to the threshold tumor heterogeneity score, predicting the subject to have a higher survival rate if treated with an anti-cancer therapy; and
if the composite tumor heterogeneity score is more than the threshold tumor heterogeneity score, predicting the subject to have a lower survival rate if treated with the anti-cancer therapy.

146. A non-transitory computer-readable storage medium storing one or more programs, the one or more programs comprising instructions, which when executed by one or more processors of a system, cause the system to:
a) obtain a medical image associated with a sample from a subject;
b) input the medical image into a trained machine learning model to identify a plurality of phenotypic features in the medical image;
c) identify a plurality of patches from the medical image;
d) identify a plurality of patch groups, wherein each patch group comprises one or more patches of the plurality of patches and corresponds to a region of interest in the medical image;
e) obtain, based on the plurality of phenotypic features, a plurality of microenvironment profiles corresponding to the plurality of patch groups; and
f) generate a phenotypic tumor heterogeneity score by comparing the plurality of microenvironment profiles.

147. The non-transitory computer-readable storage medium of clause 146, wherein the medical image comprises a histopathology image.

148. The non-transitory computer-readable storage medium of clause 146 or clause 147, further comprising instructions that, when executed by one or more processors of a system, cause the system to:
obtain a genomic tumor heterogeneity score based on sequence read data associated with the sample from the subject; and
calculate a composite tumor heterogeneity score based on the phenotypic tumor heterogeneity score and the genomic tumor heterogeneity score.

149. The non-transitory computer-readable storage medium of clause 148, wherein the composite tumor heterogeneity score is a weighted average or a binarization of the phenotypic tumor heterogeneity score and the genomic tumor heterogeneity score.

150. The non-transitory computer-readable storage medium of clause 148, further comprising: identifying a combination of treatments for the subject based on the composite tumor heterogeneity score.

151. The non-transitory computer-readable storage medium of clause 148, further comprising: determining a monitoring plan for the subject based on the phenotypic tumor heterogeneity score before sequencing the sample from the subject.

152. The non-transitory computer-readable storage medium of clause 148, further comprising:
if the composite tumor heterogeneity score is less than or equal to a threshold tumor heterogeneity score, predicting the subject to have a longer duration of therapeutic response to an anti-cancer therapy; and
if the composite tumor heterogeneity score is more than the threshold tumor heterogeneity score, predicting the subject to have a shorter duration of therapeutic response to the anti-cancer therapy.

153. The non-transitory computer-readable storage medium of clause 148, further comprising:
if the composite tumor heterogeneity score is less than or equal to the threshold tumor heterogeneity score, predicting the subject to have a higher survival rate if treated with an anti-cancer therapy; and
if the composite tumor heterogeneity score is more than the threshold tumor heterogeneity score, predicting the subject to have a lower survival rate if treated with the anti-cancer therapy.

154. A non-transitory computer-readable storage medium storing one or more programs, the one or more programs comprising instructions, which when executed by one or more processors of a system, cause the system to:
a) obtain a medical image associated with a sample from a subject;
b) identify a plurality of patches from the medical image;
c) identify a plurality of patch groups, wherein each patch group comprises one or more patches of the plurality of patches and corresponds to a region of interest in the medical image;
d) input each patch group into a trained machine learning model to generate a plurality of embeddings corresponding to the plurality of patch groups; and
e) generate a phenotypic tumor heterogeneity score by comparing the plurality of embeddings.

155. The non-transitory computer-readable storage medium of clause 154, wherein the medical image comprises a histopathology image.

156. The non-transitory computer-readable storage medium of clause 154 or clause 155, further comprising instructions that, when executed by one or more processors of a system, cause the system to:
obtain a genomic tumor heterogeneity score based on sequence read data associated with the sample from the subject; and
calculate a composite tumor heterogeneity score based on the phenotypic tumor heterogeneity score and the genomic tumor heterogeneity score.

157. The non-transitory computer-readable storage medium of clause 156, wherein the composite tumor heterogeneity score is a weighted average or a binarization of the phenotypic tumor heterogeneity score and the genomic tumor heterogeneity score.

158. The non-transitory computer-readable storage medium of clause 156, further comprising: identifying a combination of treatments for the subject based on the composite tumor heterogeneity score.

159. The non-transitory computer-readable storage medium of clause 156, further comprising: determining a monitoring plan for the subject based on the phenotypic tumor heterogeneity score before sequencing the sample from the subject.

160. The non-transitory computer-readable storage medium of clause 156, further comprising:
if the composite tumor heterogeneity score is less than or equal to a threshold tumor heterogeneity score, predicting the subject to have a longer duration of therapeutic response to an anti-cancer therapy; and
if the composite tumor heterogeneity score is more than the threshold tumor heterogeneity score, predicting the subject to have a shorter duration of therapeutic response to the anti-cancer therapy.

161. The non-transitory computer-readable storage medium of clause 156, further comprising:
if the composite tumor heterogeneity score is less than or equal to the threshold tumor heterogeneity score, predicting the subject to have a higher survival rate if treated with an anti-cancer therapy; and
if the composite tumor heterogeneity score is more than the threshold tumor heterogeneity score, predicting the subject to have a lower survival rate if treated with the anti-cancer therapy.

What is claimed is:

1. A method for determining a phenotypic tumor heterogeneity score in a sample from a subject, comprising:
obtaining a medical image associated with the sample from the subject;
identifying a plurality of patches from the medical image;
identifying a plurality of patch groups, wherein each patch group comprises one or more patches of the plurality of patches and corresponds to a region of interest in the medical image;
inputting each patch group into a trained machine learning model to generate a plurality of genomic alteration predictions corresponding to the plurality of patch groups, wherein the plurality of genomic alteration predictions is related to the presence of one or more genomic alterations in each of the input patch groups; and
generating the phenotypic tumor heterogeneity score by comparing the plurality of genomic alteration predictions.

2. The method of claim 1, wherein the medical image comprises a histopathology image.

3. The method of claim 1, further comprising:
obtaining a genomic tumor heterogeneity score based on sequence read data associated with the sample; and
calculating a composite tumor heterogeneity score based on the phenotypic tumor heterogeneity score and the genomic tumor heterogeneity score.

4. The method of claim 3, wherein the composite tumor heterogeneity score is a weighted average or a binarization of the phenotypic tumor heterogeneity score and the genomic tumor heterogeneity score.

5. The method of claim 3, further comprising: identifying a combination of treatments for the subject based on the composite tumor heterogeneity score.

6. The method of claim 3, further comprising: determining a monitoring plan for the subject based on the phenotypic tumor heterogeneity score before sequencing the sample from the subject.

7. The method of claim 3, further comprising:
if the composite tumor heterogeneity score is less than or equal to a threshold tumor heterogeneity score, predicting the subject to have a longer duration of therapeutic response to an anti-cancer therapy; and
if the composite tumor heterogeneity score is more than the threshold tumor heterogeneity score, predicting the subject to have a shorter duration of therapeutic response to the anti-cancer therapy.

8. The method of claim 3, further comprising:
if the composite tumor heterogeneity score is less than or equal to the threshold tumor heterogeneity score, predicting the subject to have a higher survival rate if treated with an anti-cancer therapy; and
if the composite tumor heterogeneity score is more than the threshold tumor heterogeneity score, predicting the subject to have a lower survival rate if treated with the anti-cancer therapy.

9. The method of claim 1, further comprising: identifying a plurality of regions of interest in the medical image.

10. The method of claim 9, wherein identifying the plurality of regions of interest comprises:
inputting the medical image into a trained segmentation machine learning model to identify a tumor tissue depicted in the medical image.

11. The method of claim 10, wherein the plurality of regions of interest corresponds to different visually distinct portions of the tumor tissue in the medical image.

12. The method of claim 10, wherein the plurality of regions of interest corresponds to different local neighborhoods of the tumor tissue in the medical image.

13. The method of claim 1, wherein the trained machine learning model is configured to receive a given input patch group and output a single prediction of the presence of a single genomic alteration in the given input patch group.

14. The method of claim 13, wherein the single genomic alteration is: an epidermal growth factor receptor (EGFR) driver mutation. a BRAF driver mutation, a MET driver mutation, an ERBB2 (HER2) driver mutation, a PIK3CA driver mutation, a KRAS driver mutation, an NRAS driver mutation, a BRCA1 driver mutation, a BRCA2 driver mutation, an ATM driver mutation, an FGFR2 driver mutation, a driver mutation in a homologous recombination repair (HRR) gene, an NTRK1 driver mutation, an NTRK2 driver mutation, an NTRK3 driver mutation, an RET driver mutation, an ALK mutation, or a ROS1 mutation.

15. The method of claim 13, wherein generating the phenotypic tumor heterogeneity score comprises:
assigning the phenotypic tumor heterogeneity score to a first value indicative of lower tumor heterogeneity if the plurality of genomic alteration predictions all indicates the presence of the single genomic alteration; and
assigning the phenotypic tumor heterogeneity score to a second value indicative of higher tumor heterogeneity if some of the plurality of genomic alteration predictions indicate the presence of the single genomic alteration and some of the plurality of genomic alteration predictions do not indicate the presence of the single genomic alteration.

16. The method of claim 13, wherein generating the phenotypic tumor heterogeneity score comprises:
calculating a variance value based on the plurality of genomic alteration predictions;
assigning the phenotypic tumor heterogeneity score to a first value indicative of lower tumor heterogeneity if the variance value is lower than or equal to a predefined threshold; and
assigning the phenotypic tumor heterogeneity score to a second value indicative of higher tumor heterogeneity if the variance value is higher than a predefined threshold.

17. The method of claim 1, wherein the trained machine learning model is configured to receive a given input patch group and output a plurality of predictions of the presence of a plurality of genomic alterations in the given patch group.

18. The method of claim 17, wherein the plurality of genomic alterations comprises: an epidermal growth factor receptor (EGFR) driver mutation. a BRAF driver mutation, a MET driver mutation, an ERBB2 (HER2) driver mutation, a PIK3CA driver mutation, a KRAS driver mutation, an NRAS driver mutation, a BRCA1 driver mutation, a BRCA2 driver mutation, an ATM driver mutation, an FGFR2 driver mutation, a driver mutation in a homologous recombination repair (HRR) gene, an NTRK1 driver mutation, an NTRK2 driver mutation, an NTRK3 driver mutation, an RET driver mutation, an ALK mutation, a ROS1 mutation, or any combination thereof.

19. The method of claim 17, wherein generating the phenotypic tumor heterogeneity score comprises:
generating a plurality of phenotypic tumor heterogeneity sub-scores corresponding to the plurality of genomic alterations, and
calculating the phenotypic tumor heterogeneity score based on the plurality of phenotypic tumor heterogeneity sub-scores.

20. A method for determining a phenotypic tumor heterogeneity score in a sample from a subject, comprising:
obtaining a medical image associated with the sample from the subject;
inputting the medical image into a trained machine learning model to identify a plurality of phenotypic features in the medical image;
identifying a plurality of patches from the medical image;
identifying a plurality of patch groups, wherein each patch group comprises one or more patches of the plurality of patches and corresponds to a region of interest in the medical image;
obtaining, based on the plurality of phenotypic features, a plurality of microenvironment profiles corresponding to the plurality of patch groups; and
generating the phenotypic tumor heterogeneity score by comparing the plurality of microenvironment profiles.
